(19) United States
(12) Patent Application Publication
(10) Pub. No.: US 2026/0055330 A1
(43) Pub. Date: Feb. 26, 2026

(54) HIGH ENERGY DENSITY ROCKET FUEL COMPOSITIONS

(71) Applicant: Visolis, Inc., Hayward, CA (US)

(72) Inventors: Deepak DUGAR, Dallas, TX (US); Shylesh Sankaranarayanapilla, Albany, CA (US)

(21) Appl. No.: 19/281,680

(22) Filed: Jul. 27, 2025

Related U.S. Application Data
(60) Provisional application No. 63/675,805, filed on Jul. 26, 2024.
(60) Provisional application No. 63/726,561, filed on Dec. 1, 2024.

(51) Int. Cl. C10L 1/04; C07C 1/213; C07C 2/46; C07C 2/50; C10G 3/00

(52) U.S. Cl. CPC C10L 1/04; C07C 1/213; C07C 2/46; C07C 2/50; C10G 3/42; C10G 2400/08; C10L 2200/043

(57) ABSTRACT

The invention is related to a process for synthesizing a sustainable aviation fuel composition. The process comprises providing a reaction mixture comprising a first compound that is at least one of mevalonolactone, mevalonic acid, mevalonate salt, dehydromevalonic acid, dehydromevalonate salt, levulinic acid, levulinate salt, caproic acid, caprolactone, caproate salt, or combinations thereof. The process then involves converting the first compound in the reaction mixture to provide a first intermediate comprising isoprene. Then, the isoprene in the first intermediate is reacted in the presence of a first heat transfer agent to provide a second intermediate comprising terpenes. Finally, the second intermediate is allowed to react to in the presence of a second heat transfer agent, or alternatively in neat conditions, and optionally in presence of a catalyst to provide the sustainable aviation fuel composition. The sustainable aviation fuel composition made available from the process of the invention is found to be substantially devoid of Sulphur, olefins, MAHs and PAHs while retaining high energy density. Such qualities are deeply appreciated for use of such sustainable aviation fuel compositions as rocket propellants.

This is a complete specification for the Provisional Application No. 63/675,805 filed on Jul. 26, 2024 and the Provisional Application No. 63/726,561 filed on Dec. 1, 2024.

### TECHNICAL FIELD OF THE INVENTION

The invention generally relates to a composition for fuel blending, and more specifically to a composition that is made by a process for reacting a reactant mixture at a reaction temperature.

### BACKGROUND

The traditional routes for synthesizing specialized and commodity chemicals are typically specific for each chemical, and rarely adaptable for other chemicals. Typically, these chemicals are synthesized in a process involving multiple steps. Some of the exemplary reactions involved in the synthesis include ring-opening metathesis reactions, ring-closing metathesis reactions, Diels-Alder reactions, isomerizations, decarboxylations, hydrogenations, and so on. However, in many cases at least one or more of the steps of these processes are not made in a sustainable manner. Sustainability is addressed in a number of different ways, ranging from the starting materials, the processes used for the manufacture, all the way to composition of the final product including any additives and traces of impurities present in them, to the manner in which these products are used.

One of the ways of defining sustainability includes the choice of starting materials from biological processes and/or renewable resources for several different reasons. Economic factors are one of the chief drivers for the choice of raw materials from sustainable sources as they are made from renewable feedstocks where the resources are often utilized very efficiently. From a technological standpoint, such sustainable materials are made from processes that are atom efficient that make the processes cost effective. Further, sustainable materials can help reduce harmful emissions and minimize waste, making processes more eco-friendly. In many countries, local regulations have been implemented or will be imminently implemented that will require use of such sustainable materials to allow industries to remain compliant or stay ahead of the curve when such regulations are implemented.

There are some sustainable chemistries used in a wide variety of reactions, including for example metathesis reactions and Diels-Alder reactions. These reactions are known to have excellent atom economy, and can use starting materials from renewable sources. However, whilst the reactions involving sustainable chemistries often work well for relatively small molecules, the reactions are often not so specific when synthesizing larger sized molecules, resulting in a number of by-products. Further, the nature of these by-products can be uncontrolled. This situation is often encountered while synthesizing sustainable aviation fuels, wherein a significant fraction of the products includes monoaromatic hydrocarbons and that is associated with a significant amount of by-product polyaromatic hydrocarbons. It is known that monoaromatic hydrocarbons at up to certain concentration levels in the aviation fuels play an important role by imparting necessary seal swelling properties, however, polyaromatic hydrocarbons lead to particulate formation when they are burnt. This in turn promotes the formation of contrails. Indeed, this is encoded in the regulations for aviation fuels that indicate the maximum amount of monoaromatic and polyaromatic hydrocarbons that can be present in the fuel.

Thus, in many cases the polyaromatic hydrocarbon content can be too high and in that case, the monoaromatics need to be separated from the polyaromatics, which would require additional steps in the production. It would be advantageous to have a synthetic process that produces the monoaromatics almost exclusively while limiting the undesirable polyaromatics in the final product to less than the allowable levels, or preferably to no polyaromatics at all.

Also, most of the reactions result in impurities occurring in the final product, even at trace levels, that impart suboptimal properties to the final product. This may result in the products being rendered useless for some applications. Overcoming these problems would require extensive post reaction purification or separation steps, which may not be economically feasible and thus most often, these suboptimal products are sold in the market. An example is the presence of sulfur in fuels at levels that result in considerable environmental pollution when the fuel is burnt. Similarly sulfur compounds can act as chain terminators in polymerization reactions and are thus undesirable even at low levels in polymerization applications.

U.S. Pat. No. 9,650,315B1 describes a method for conversion of isoprene to full performance jet and diesel fuels. Isoprene can be selectively oligomerized to generate a distribution of branched chain hydrocarbons. Combination of an oligomerization catalyst with a metathesis catalyst allows for the synthesis of high density cyclic fuels with performance advantages (increased density and volumetric net heat of combustion) over conventional petroleum-based fuels. U.S. Pat. No. 9,682,897B1 discloses a method for the efficient synthesis of useful deoxygenated terpenoids from an abundant renewable source, using catalytic conversion of oxygenated terpenoids that are made available from renewable resources (for example, 1,4-cineole and 1,8-cineole are major components of turpentine and essential oils). Catalytic deoxygenation of these substrates can be used to efficiently generate commercially important chemicals and high density fuels for turbine or diesel propulsion.

US2016289573A1 for example discloses a process for the production of hydrocarbon fluids having a low aromatic and sulfur content. In this process, all types of aromatic content is significantly reduced in the final product. Similarly, US2009173665A1 relates to catalysts and processes for increasing the monoaromatic content of hydrocarbon feedstocks, including polynuclear aromatic hydrocarbons, wherein the increase in monoaromatics can be achieved by increasing the gasoline/diesel yield and simultaneously reducing unwanted compounds, thereby a route for upgrading hydrocarbons is provided that includes a significant amount of polynuclear aromatic hydrocarbons. In this prior art, the polyaromatic hydrocarbons are converted to monoaromatic hydrocarbons in a separate step, thus increasing the number of steps in the production and increasing costs of production. WO 2012133138 also refers to the use of a diluent making it possible to reduce the exothermicity of the hydrogenation reaction of polycyclic aromatics of a heavy petroleum cut obtained by catalytic reforming which is then reformed again in order to obtain a monoaromatic hydrocarbon-containing cut. U.S. Pat. No. 10,927,311B2 discloses a process for the dearomatization of a petroleum cut to produce a dearomatized hydrocarbon-containing fluid having a very low sulfur content and having a very low content of aromatic compounds. All the prior art disclose processes involving increasing complexity with the consequent economic costs.

One significant feature of sustainable chemistries involves the coke formation that leads to wasted materials, loss in efficiency, labor intensive and cost intensive steps to clean out the reactor. Eleni Heracleous et al in ACS Catal. 2020, 10, 16, 9649-9661 highlights a critical problem faced while using sustainable chemistries for synthesizing starting materials such as isoprene, terpenes, terpinolenes etc. In that paper, it was clearly proven that running such reactions in reactors for extended periods of time resulted in considerable coke formation. This had to be addressed very frequently, which resulted in shutting down the reactor for maintenance and cleaning up operations. This would generally mean that such reactions are not very conducive for Continuous Flow Reactors for longer periods of time as it would result in clogging of inlets, outlets and other locations in the reactors. While one skilled in the art may be drawn towards designing alternate chemistries to address these issues, the advantages provided by the existing chemistries in terms of atom efficiency, mass balance, yields, selectivity are worth pursuing to achieve at least near theoretical levels. However, problems like the coke formation continue to plague the existing sustainable chemistries processes thus the tremendous potential of these processes continues to not be achieved.

Thus, there is a need to develop efficient processes that provide products at high yields with greater specificity and better control over the byproducts from the processes in the final product.

### BRIEF DESCRIPTION OF THE INVENTION

In one aspect, the invention provides a reactant mixture for conducting multistep reactions to produce products with high selectivity and yields. The reactant mixture comprises a first compound comprising at least one carbon-carbon double bond or a precursor therefor; a heat transfer fluid; and at least one stabilizer. The invention also provides for a product made from a process using the reactant mixture of the invention.

In another aspect, the invention provides a composition for fuel blending, wherein the composition is made by a process for reacting the reactant mixture as described herein at a reaction temperature, wherein the composition for fuel blending is substantially free of C₇ hydrocarbons. The invention also provides a fuel composition comprising the composition for fuel blending.

In a further aspect, the invention provides a sustainable aviation fuel composition comprising monocyclic aromatic hydrocarbons (MAHs) in a concentration ranging from about 1% by volume to about 25% by volume; and polycyclic aromatic hydrocarbons (PAHs) in a concentration ranging from about 0% by volume to about 5% by volume. The sustainable aviation fuel composition is advantageously made by the process for reacting the reactant mixture as described herein.

In a yet another aspect, the invention provides a process for synthesizing the sustainable aviation fuel composition. The process comprises providing a reaction mixture comprising a first compound that is at least one of mevalonolactone, mevalonic acid, mevalonate salt, dehydromevalonic acid, dehydromevalonate salt, levulinic acid, levulinate salt, caproic acid, caprolactone, caproate salt, or combinations thereof. The process then involves converting the first compound in the reaction mixture to provide a first intermediate comprising isoprene. Then, the isoprene in the first intermediate in the presence of a heat transfer agent to provide a second intermediate comprising terpenes; and finally reacting the second intermediate in the presence of the first heat transfer agent or alternatively, a second heat transfer agent, and optionally a catalyst to provide the sustainable fuel composition.

In another embodiment, the invention provides a process for synthesizing a sustainable aviation fuel composition. The process comprises providing a first intermediate mixture comprising a first compound that comprises at least one carbon-carbon double bond. The first compound comprising the olefinic bond is then reacted in the presence of a first heat transfer agent to provide a second intermediate; and finally reacting the second intermediate in the presence of a second heat transfer agent to provide the sustainable fuel composition.

In yet another aspect, the invention provides a rocket fuel composition comprising alicyclic hydrocarbons and optionally aromatic hydrocarbons wherein the total renewable content is at least 5% w/w, preferably 10% w/w, preferably 15% w/w, preferably 20% w/w, preferably 25% w/w, preferably 30% w/w of the composition.

In yet another aspect, the invention provides a monomer composition comprising unsaturated hydrocarbons wherein the total renewable content is 5% by weight, preferably 10% by weight, preferably 15% by weight, preferably 20% by weight, preferably 30% by weight, preferably 40% by weight, preferably 50% by weight, preferably 60% by weight, preferably 70% by weight, preferably 80% by weight, preferably 90% by weight, preferably 95% by weight of the composition and where chain terminating compounds such as mercaptans are substantially absent.

In a further aspect, the invention provides a feedstock and a process for the synthesis of tackifier compositions wherein the feedstock is made from a renewable source and comprises terpenes or precursors thereof.

In a further aspect, the invention provides a feedstock and a process for the synthesis of homopolymers, copolymers, block copolymers, graft copolymers, wherein the feedstock is made from a renewable source and comprises isoprene, terpenes, or precursors thereof.

### BRIEF DESCRIPTION OF DRAWINGS

FIG. 1 is a proton NMR spectrum of the cycloalkanes obtained from fractional distillation as described in example 18.

FIG. 2 is a carbon-13 NMR spectrum of the cycloalkanes obtained from fractional distillation as described in example 18.

FIG. 3 is a plot of the number of carbons versus the percent mass of the fuel composition described in example 24.

FIG. 4 is a distillation curve of the fuel composition described in example 23.

FIG. 5 a plot of the number of carbons versus the percent mass of the fuel composition made by a different solvent ratio as described in example 24 d compared to the distillation curve of a sample of Jet A aviation fuel.

FIG. 6 is a distillation curve of the fuel composition made using a different solvent ratio as described in example 24 shown in comparison to a sample of Jet A aviation fuel.

FIG. 7 shows an image of the catalyst layer from the reactor after a reaction that clearly shows coke formation.

FIG. 8 shows various physical properties were measured for the fuel compositions as described in examples 14, 26 and 27.

FIG. 9 shows a graph of the seal swell properties as a function of the aromatic content, as compared with those of typical jet fuels.

### DETAILED DESCRIPTION

As used herein and in the claims, the singular forms “a,” “an,” and “the” include the plural reference unless the context clearly indicates otherwise.

As used herein, the term “fuel” is any material that can be combusted to release energy as thermal energy that can then be converted into mechanical energy via a heat engine. Hydrocarbons and related organic molecules having a suitable carbon-to-hydrogen ratio provide the appropriate levels of energy content are by far the most suitable fuels.

As used herein, and unless otherwise specified, the term “renewable fuel” refers to any fuel derived from a biological source or biomass through processes such as, but not limited to hydrotreating, thermal conversion and biomass-to-liquid.

“Fuel blend” refers to mixtures of traditional fuels such as gasoline, diesel, aviation fuel, and alternative fuels such as hydrocarbons obtained from sustainable processes in varying percentages.

Seal swelling refers to an increase in a volume of an elastomer seal when it absorbs gas or liquid. Seal swelling properties may be measured based on standardized testing methods, such as, for example ASTM D471 (2021).

Carbon atoms of renewable origin comprise a higher number of C-14 isotopes compared to carbon atoms of fossil origin. Therefore, it is possible to distinguish the hydrocarbons of renewable origin (such as in a renewable hydrotreated fuel or in a renewable polymer composition) from non-renewable hydrocarbons (such as those derived from fossil fuels) by analyzing the amount of C-14 isotopes. The amount of a renewable fuel in a blend can be determined according to for example ASTM D6866 (2018).

As used herein the term “aliphatic” or “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, halo alkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl; difluorovinylidene; trichloromethyl, bromodichloromethyl, bromoethyl, 2-bromotrimethylene (e.g. —CH₂CHBrCH₂—), and the like. Further examples of aliphatic radicals include allyl, aminocarbonyl (i.e. —CONH₂), carbonyl, dicyanoisopropylidene (i.e. —CH₂C(CN)₂CH₂—), methyl (i.e. —CH₃), methylene (i.e. —CH₂—), ethyl, ethylene, formyl (i.e. —CHO), hexyl, hexamethylene, hydroxymethyl (i.e. —CH₂OH), mercaptomethyl (i.e. —CH₂SH), methylthio (i.e. —SCH₃), methylthiomethyl (i.e. —CH₂SCH₃), methoxy, methoxycarbonyl (i.e. CH₃OCO—), nitromethyl (i.e. —CH₂NO₂), thiocarbonyl, trimethylsilyl (i.e. (CH₃)₃Si—), t-butyldimethylsilyl, trimethoxysilylpropyl (i.e. (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e. CH₃—) is an example of a C₁ aliphatic radical. A decyl group (i.e. CH₃(CH₂)₉—) is an example of a C₁₀ aliphatic radical.

As used herein, the term “aromatic” or “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic” or “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthracenyl groups (n=3) and the like. The aromatic radical may also include nonaromatic components. For example, a benzyl group is an aromatic radical which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as trifluoromethylphenyl, hexafluoroisopropylidenebis(4-phen-1-yloxy) (i.e. —OPhC(CF₃)₂PhO—), chloromethylphenyl; 3-trifluorovinyl-2-thienyl; 3-trichloromethylphen-1-yl (i.e. 3-CCl₃Ph-), 4-(3-bromoprop-1-yl)phen-1-yl (i.e. BrCH₂CH₂CH₂Ph-), and the like. Further examples of aromatic radicals include 4-allyloxyphen-1-oxy, 4-aminophen-1-yl (i.e. H₂NPh-), 3-aminocarbonylphen-1-yl (i.e. NH₂COPh-), 4-benzoylphen-1-yl, dicyanoisopropylidenebis(4-phen-1-yloxy) (i.e. —OPhC(CN)₂PhO—), 3-methylphen-1-yl, methylenebis(phen-4-yloxy) (i.e. —OPhCH₂PhO—), 2-ethylphen-1-yl, phenylethenyl, 3-formyl-2-thienyl, 2-hexyl-5-furanyl; hexamethylene-1,6-bis(phen-4-yloxy) (i.e. —OPh(CH₂)₆PhO—); 4-hydroxymethylphen-1-yl (i.e. 4-HOCH₂Ph-), 4-mercaptomethylphen-1-yl (i.e. 4-HSCH₂Ph-), 4-methylthiophen-1-yl (i.e. 4-CH₃SPh-), 3-methoxyphen-1-yl, 2-methoxycarbonylphen-1-yloxy (e.g. methyl salicyl), 2-nitromethylphen-1-yl (i.e. -PhCH₂NO₂), 3-trimethylsilylphen-1-yl, 4-t-butyldimethylsilylphenyl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₈—) represents a C₇ aromatic radical.

As used herein the term “cycloaliphatic” or “cycloaliphatic radical” or “alicyclic radical” or “alicyclic” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is a cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, halo alkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene2,2-bis (cyclohex-4-yl) (i.e. —C₆H₁₀C(CF₃)₂C₆H₁₀—), 2-chloromethylcyclohex-1-yl; 3-difluoromethylenecyclohex-1-yl; 4-trichloromethylcyclohex-1-yloxy, 4-bromodichloromethylcyclohex-1-ylthio, 2-bromoethylcyclopent-1-yl, 2-bromopropylcyclohex-1-yloxy (e.g. CH₃CHBrCH₂C₆H₁₀—), and the like. Further examples of cycloaliphatic radicals include 4-allyloxycyclohex-1-yl, 4-aminocyclohex-1-yl (i.e. H₂NC₆H₁₀—), 4-aminocarbonylcyclopent-1-yl (i.e. NH₂COCH₈—), 4-acetyloxycyclohex-1-yl, 2,2-dicyanoisopropylidenebis(cyclohex-4-yloxy) (i.e. OC₆H₁₀C(CN)₂C₆H₁₀O—), 3-methylcyclohex-1-yl, methylenebis(cyclohex-4-yloxy) (i.e. —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl; hexamethylene-1,6-bis(cyclohex-4-yloxy) (i.e. —OC₆H₁₀(CH₂)₆C₆H₁₀O—); 4-hydroxymethylcyclohex-1-yl (i.e. 4-HOCH₂C₆H₁₀—), 4-mercaptomethylcyclohex-1-yl (i.e. 4-HSCH₂C₆H₁₀—), 4-methylthiocyclohex-1-yl (i.e. 4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e. NO₂CH₂C₆H₁₀—), 3-trimethylsilylcyclohex-1-yl, 2-butyldimethylsilylcyclopent-1-yl, 4-trimethoxysilylethylcyclohex-1-yl (e.g. (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein, “polycyclic aromatic hydrocarbon” (PAH) or “polynuclear aromatic hydrocarbon” (PNA) is a class of organic compounds that is composed of multiple aromatic rings. Simple representative PAHs include naphthalene, having two fused aromatic rings, biphenyl wherein two phenyl rings are connected to each other and the three-ring compounds anthracene and phenanthrene. PAHs are generally uncharged and non-polar. By definition, PAHs have multiple aromatic rings, thus precluding benzene from being considered a PAH.

“Terpenes” as used herein means a class of compounds having a generic formula (C₅H₈)ₙ, wherein n≥2, and generally characterized by having at least one double bond. Terpenes are classified by the number of carbons: monoterpenes (C₁₀), sesquiterpenes (C₁₅), diterpenes (C₂₀), as examples. When the terpenes are substituted, one of the hydrogens is replaced with the substituent group, however, the double bond characteristics that would render the compound conducive for further reactions as described herein would remain intact.

Certain types of material referred to herein, such as, for example, hydrogenated esters and fatty acids, are derived from processes that do not always generate a pure compound but instead generate a range of materials within a set of particular physical boundaries, for example boiling range. As used herein, we sometimes refer to the main component of such a mixture.

As used herein, a “12 carbon aliphatic hydrocarbon” means that the 12 carbon aliphatic hydrocarbon is the most predominant component, and other compounds such as for example 12 carbon aromatic hydrocarbons, 10 carbon aliphatic hydrocarbons, 14 carbon alicyclic hydrocarbons may also be present. The exact concentration that renders a particular compound as being predominant depends on various factors such as, but not limited to, final application, desired properties, concentration of other compounds, synthetic processes involved, and the like, and combinations thereof. Thus, in some embodiments, greater than 90% by weight may be considered to be predominant concentration; in other embodiments, greater than about 80% by weight may be considered to be predominant concentration; in yet other embodiments, greater than about 60% by weight may be considered to be predominant concentration; in yet other embodiments, greater than about 40% by weight may be considered to be predominant concentration. It would be understood that predominant concentration means that other compounds are individually present in significantly lower concentrations. The exact values and concentrations that render a particular compound as predominant or as insignificant would become apparent to one skilled in the art.

It should be understood that when such a mixed composition is then further combined with other materials to form a new composition, it is perfectly possible that the predominant component of the material in the feed composition may not be a predominant material in the final composition by virtue of the relative quantities used.

Unsaturated hydrocarbons are a major category of chemical product with many varied uses. In particular they find uses as chemical intermediates, as monomers in the production of oligomers and polymers and as functional molecules with applications such as solvents or fuels. There are a variety of methods for producing or extracting unsaturated hydrocarbons and their derivative compounds. It is, however, often the case that such production processes result in products which are less than ideal in terms of economics, composition or environmental sustainability. The present invention generally relates to reactant compositions and methods for producing certain unsaturated hydrocarbons and derivatives thereof. It brings advantages in improving yield, reducing certain unwanted by-products and in lending itself to being performed using renewable feedstocks, thus addressing the aforementioned challenges with existing processes. In one aspect, the invention is related to a reactant mixture for conducting multistep reactions that surprisingly has been found to obtain products with high selectivity and yields in single or multi step reactions. The reactant mixture comprises a first compound comprising at least one carbon-carbon double bond or a precursor therefor, a heat transfer fluid, and at least one stabilizer.

The first compound is at least one of isoprene, monoterpenes, sesquiterpenes, diterpenes, polyterpenes, or precursors of isoprene, monoterpenes, sesquiterpenes, diterpenes, polyterpenes. The first compound may be obtained from a variety of sources, including from fossil fuel sources or from biological processes. In a preferred embodiment, the first compound is a product from sustainable processes. Suitable precursors of the isoprene or various types of terpenes that can be used as the first compound of the reaction mixture includes at least one of mevalonolactone, mevalonic acid, mevalonate salt, dehydromevalonic acid, dehydromevalonate salt, levulinic acid, levulinate salt, caproic acid, caprolactone, caproate salt, or combinations thereof. It is known that mevalonic acid compounds can exist in its lactone form, salt form and free acid form under suitable conditions. Thus, the use of one of the compounds automatically implies the presence of the other compounds unless the reaction mixture is designed to specifically suppress one or the other form of the compound. One skilled in the art will be able to recognize which of the forms of the compound will be present in the reaction mixture based on the reaction conditions used. For example, without being bound to any theory, it is known that the presence of appropriate counterions such as Na, K, Ca, Ba etc. in mevalonate salt would increase the evolution of carbon dioxide under appropriate reaction conditions thus making it a suitable starting material for decarboxylation reaction.

The first compound may further comprise at least one of: a conjugated diene, one or more other types of olefins, isoprene, butadiene, C₃₋₁₀ olefins, other diene compounds, and the like, and combinations thereof.

The heat transfer fluid is an inert reaction medium having properties such as, but not limited to, a minimum heat capacity of 1 kJ/kg-K (at operating conditions), minimum thermal conductivity of 0.1 W/m-K (at operating conditions), and a maximum kinematic viscosity of 10 Centistoke (at operating conditions), high boiling point or boiling range greater than 100 degrees Centigrade at standard pressure, preferably greater than 110° C., preferably greater than 130 degrees Centigrade, preferably greater than 150 degrees Centigrade, preferably greater than 170 degrees Centigrade, preferably greater than 200 degrees Centigrade, preferably greater than 250 degrees Centigrade. The heat transfer fluid may comprise petroleum distillates, hydrotreated heavy paraffinic compounds, white mineral oil, and the like, and combinations thereof. The heat transfer fluid may further comprise Dimethyl Siloxane, Trimethylsiloxy terminated siloxane. Specific examples of heat transfer fluid include the chemical compound represented by CAS #8042-47-5, commercially made available from Duratherm Inc, Lewiston, NY, CAS #63148-62-9, etc. Other heat transfer fluids include, for example, but not limited to, the Paratherm line of heat transfer fluids, the Xceltherm line of heat transfer fluids, the Dowtherm heat transfer fluids, and the like, and combinations thereof. Each of these lines of heat transfer fluids is commercially readily available from the respective commercial provider. The heat transfer fluids are also known to be quite stable and can be recycled several times without affecting the reaction yields, selectivity, efficiency, and other reaction parameters. Further for some applications such as fuels, certain heat transfer fluids may be part of the final product as they do not affect the final properties of the product, or in some instances they contribute to improve the final properties. Ratio of the first compound to the heat transfer fluid in the reactant mixture varies from about 1:1000 to 10:1 by weight.

Heat transfer fluids may also include suitable molten salts that remain liquids within a desired temperature range, for example 120 C to about 650 C. Exemplary molten salts useful in the invention may include, but are not limited to, nitrates such as lithium nitrate, sodium nitrate, potassium nitrate, carbonates such as calcium carbonates, sodium carbonate, potassium carbonate, and the like, and combinations thereof. In some instances, the molten salts may be a eutectic mixture of two or more salts, such as sodium nitrate and potassium nitrate. Such molten salts are also commercially available from sources such as Dynalene MS-1 or MS-2 made available from Dynalene, Inc. or Globaltherm® Omnistore MS-600 made available from Global Heat Transfer Ltd.

Heat transfer fluids (HTFs) are quite stable over a wide temperature range. Heat transfer fluids allow for rapid removal of products formed quickly as the reactions are conducted at a temperature above the boiling point of the products. The rapid removal of the products ensures no further reactions occur, thus ensuring greater conversion to desired products. HTFs also allow for precise thermal control, which would ensure that there are no localized hot spots leading to runaway reactions, even for highly exothermic reactions such as Diels-Alder reaction. These factors directly translate into higher selectivity in the reaction. Heat transfer fluids may also be sometimes referred to as heat transfer agents.

The use of heat transfer fluids also prolongs the life of any catalysts being used for the various reactions. Without being bound to theory, it has been observed that under certain conditions, the catalysts may become covered with a layer of coke, which in turn reduces catalytic activity (See for example Efficient Route for the Production of Isoprene via Decarboxylation of Bioderived Mevalonolactone. Eleni Heracleous, Eleni Pachatouridou, Lin Louie, Deepak Dugar, and Angelos A. Lappas. ACS Catalysis 2020 10 (16), 9649-9661.) The coke formation may lead to further problems such as clogging the reactors, leading to difficulty in cleaning the reactors. This problem is enhanced in some reactor types such as continuous flow reactors. The reactant mixture of the invention has been found to surprisingly reduce coke formation while conducting different types of reactions over a long period of time, and also when the catalysts are reused over several cycles of reaction.

The at least one stabilizer in the reaction mixture can be for example an amine, preferably an aromatic amine such as benzeneamine, N-phenyl benzene amine, or combinations thereof. Generally, the reaction mixture comprises the at least one stabilizer in a concentration ranging from about 0.005 wt % to about 5 wt %.

The reactant mixture can further comprise a polymerization inhibitor to inhibit any potential homopolymerization and/or copolymerization of the reactants and/or products that contain olefinic bonds that are otherwise prone to polymerize. Exemplary polymerization inhibitors useful in the invention include, for example, but not limited to, tert-butyl catechol, dintrocresols such as 4,6-dinitrol-o-cresol (DNCO), hydroquinone, mequinol, and combinations thereof. The polymerization inhibitor is made available at a concentration ranging from about 10 ppm to about 4000 ppm.

At least one of the heat transfer fluid, stabilizer, or the polymerization inhibitor can be recycled from a previous reaction.

The reactant mixture can further comprise a catalyst that catalyzes the respective reaction. Suitable catalysts for specific reactions are well known to those skilled in the art. Exemplary catalysts include a solid catalyst such as metal oxide or mixed metal oxide or non-metal oxide catalysts such as silicon oxide, titanium dioxide, zirconium oxide, aluminium oxide, niobium oxide, cerium oxide, tin oxide, silica-alumina, tungstated zirconia, sulfated zirconia, silica or alumina doped with metals such as palladium, ruthenium, rhenium, tantalum, manganese, vanadium, chromium, nickel, iron, cobalt, copper, silver, tin, gold, and mixtures thereof; montmorillonites such as montmorillonite K-10 and montmorillonite K-30; zeolites such as zeolite X, zeolite Y, zeolite L (faujasite, chabazite, mordenite, ZSM-5, H-BEA); Nafion SAC-13, Nafion NR50, Amberlyst-15, Amberlyst-45, silica supported sulphonic acid, niobium phosphate, or combinations thereof. Catalysts useful in the invention may also be recycled from previous reactions based on their activity and Turnover Number (TON) or Turnover Frequency (TOF). Catalysts may also comprise a hydrogenation catalyst that is a single atom hydrogenation catalyst.

Using the reactant mixture described herein, suitable products can be made through appropriate processes that depend on the starting materials and the desired products. In some embodiments, that comprise heating the reactant mixture to a temperature ranging from about 50° C. to about 500° C. the processes of the invention can be used for a wide variety of reactions, such as, but not limited to, dehydration reaction, isomerization reaction, decarboxylation reaction, dehydrogenation reaction, and so on, and combinations thereof. It has been found that the processes involving the reactant mixture of the invention can be used for multi-step single pot reactions to produce products with high selectivity and yields.

In some embodiments, the product is comprised entirely of aromatic compounds. In other embodiments, the product comprises at least about 30 wt % aromatic compounds, preferably at least about 25 wt % aromatic compounds, or preferably at least about 20 wt % aromatic compounds, or preferably at least about 15 wt % aromatic compounds, or preferably at least about 10 wt % aromatic compounds, or preferably at least about 5 wt % aromatic compounds.

In yet other embodiments, the product is comprised entirely of alicyclic compounds. In other embodiments, the product comprises at least about 30 wt % alicyclic compounds, preferably at least about 25 wt % alicyclic compounds, or preferably at least about 20 wt % alicyclic compounds, or preferably at least about 15 wt % alicyclic compounds, or preferably at least about 10 wt % alicyclic compounds, or preferably at least about 5 wt % alicyclic compounds.

The products useful in the invention may have characteristics and properties required of certain use case scenarios. In some specific embodiments, the product has a density above 0.78 g/cm³ at 20° C. and a flash point greater than 38° C.

Suitable products that can be obtained using the reactant mixture include, but are not limited to, 1-butene, 2-butene, isoprene, monoterpenes, sesquiterpenes, diterpenes, polyterpenes, acrolein, acrylic acid, hydroxymethyl furfural, dipentene, limonene, carvestrene, 4-ethenyl-1,4-dimethyl cyclohexene, terpinolenes, dimethyl cyclooctadienes, 4-ethenyl-1,4-dimethyl cyclohexene, dimethyl cyclooctadienes, 3-methyl-1,3-butadiene, [symbol] -terpinene, ©-terpinene, terpinolenes, isoterpinolene, isolimonene, isocarvestrene, p-cymene sesquiterpenes, diterpenes, bicyclic alkanes or combinations thereof.

Further products are achievable by adding additional olefinic reactants. For example, adding n-alkenes to a reactant mixture comprising isoprene allows the production of 1-methyl-4-alkylcyclohex-1-ene(s) and 2-methyl-4-alkylcyclohex-1-ene(s).

Likewise adding n-alkenes to a reactant mix comprising butadiene allows the production of 4-alkylcyclohex-1-enes as well as 4-vinylcyclohex-1-ene.

Sources of additional olefins include:
a) Individual olefins derived from ethylene oligomerization such as 1-hexene, 1-octene.
b) Mixed olefins derived from ethylene oligomerization such as a mix of butene, hexene, octene and decene.
c) Mixed olefins derived or accessed from the Fischer Tropsch (FT) style oligomerization process directly or indirectly from longer chain FT hydrocarbons via a combination of cracking, distillation and dehydrogenation such as a mix of C₃ to C₁₀ olefins.
d) Mixed olefins derived from a combination of cracking, distillation and dehydrogenation of hydroprocessed esters and fatty acids such as a mix of C₃ to C₁₀ olefins.
e) Olefins derived from dehydrogenation of natural gas liquids such as C₃ to C₆ olefins.

In some specific embodiments, the reaction mixture of the invention comprises mevalonolactone (or its acid or salt form) derived from various biochemical routes and hence is a sustainable source of raw material. The mevalonolactone is converted to isoprene as described herein.

The isoprene made from the reactant mixture of the invention is then used to make useful compounds and products, wherein the final products are substantially devoid of impurities that are typically associated with isoprene obtained from other sources such as biological production or petroleum sources. Thus, for example, the isoprene is used to make polymers comprising isoprene repeat units comprising less than 10 ppm of sulfur, preferably less than 5 ppm of sulfur, preferably less than 3 ppm of sulfur, preferably less than 1 ppm of sulfur, preferably less than 0.5 ppm of sulfur. The polymer is also substantially devoid of C5 compounds, such as a C5 prenyl alcohol. Other impurities associated with isoprene production, and consequently present in the final product include singly or in combinations thereof, for example, but not limited to, ethanol, acetone, methanol, acetaldehyde, methacrolein, methyl vinyl ketone, 3-methylfuran, 2-methyl-2-vinyloxirane, cis- and trans-3-methyl-1,3-pentadiene, a C5 prenyl alcohol (such as 3-methyl-3-buten-1-ol or 3-methyl-2-buten-1-ol), 2-heptanone, 6-methyl-5-hepten-2-one, 2,4,5-trimethylpyridine, 2,3,5-trimethylpyrazine, citronellal, methanethiol, methyl acetate, 1-propanol, diacetyl, 2-butanone, 2-methyl-3-buten-2-ol, ethyl acetate, 2-methyl-1-propanol, 3-methyl-1-butanal, 3-methyl-2-butanone, 1-butanol, 2-pentanone, 3-methyl-1-butanol, ethyl isobutyrate, 3-methyl-2-butenal, butyl acetate, 3-methylbutyl acetate, 3-methyl-3-buten-1-yl acetate, 3-methyl-2-buten-1-yl acetate, (E)-3,7-dimethyl-1,3,6-octatriene, (Z)-3,7-dimethyl-1,3,6-octatriene, (E,E)-3,7,11-trimethyl-1,3,6,10-dodecatetraene and (E)-7,11-dimethyl-3-methylene-1,6,10-dodecatriene, 3-hexen-1-ol, 3-hexen-1-yl acetate, limonene, geraniol (trans-3,7-dimethyl-2,6-octadien-1-ol), citronellol (3,7-dimethyl-6-octen-1-ol), (E)-3-methyl-1,3-pentadiene, (Z)-3-methyl-1,3-pentadiene. All these impurities are generally known in the art to be bio-byproduct, by-products of fermentation process. Similar such byproduct impurities from petroleum based processes are also known in the art. In the absence of the reactant mixture of the invention, isoprene produced from the prior art reactants and processes are subjected to repeated purification steps, resulting in labor intensive and costly processes.

The polymer derived from isoprene may include homopolymers or copolymers or block copolymers, or random copolymers, or graft copolymers. Polymerization of isoprene with or without comonomers can be effected using known methods in the art, such as for example, free radical polymerization, ionic polymerization, and the like. Exemplary comonomers useful to make copolymers, include for example, but not limited to, styrene, butadiene, and the like. Thus, the invention provides polymers such as, but not limited to, butadiene-isoprene rubbers, styrene-isoprene copolymer rubbers, styrene-isoprene-butadiene rubbers, styrene-isoprene-styrene block copolymers, and styrene-isoprene block copolymers, that are substantially devoid of C5 and/or bio-byproduct impurities. Polymers comprising isoprene repeat units may have number average molecular weights ranging from about 10,000 to about 1,000,000.

The isoprene, alkenes, terpenes, terpinenes etc. from the reaction mixture or obtained from other sources are then subjected to Diels-Alder condensation reaction with other suitable reactants to yield terpenes such as limonene, carvestrene, terpinenes, terpinolenes, 4-ethenyl-1,4-dimethylcyclohexene, 2-methyl-1,3-butadiene, and the like, in high yields. Alternately, the isoprene is subjected to self-condensation in the Diels-Alder reaction to yield suitable products. In certain embodiments, water is used as a co-solvent since water can exhibit rate enhancement in Diels-Alder reactions due to hydrogen bonding effect at the transition state. Other polar solvents that may be used include, for example linear alcohols, branched alcohols, cyclic alcohols, ethers, esters, and any other polar group known in the art. Mixtures of water and these solvents may also be used.

It is known in the art that synthesizing compounds such as limonene and carvestrene in high yields and high selectivity is difficult. Some of the reactions described in the art are not conducive for large scale industrial production as they are prone to side reactions leading to undesirable by-products. Thus, in one aspect, this invention addresses this problem by providing reactant mixtures and methods for such synthesis that provide products in high yields and specificity while minimizing side reactions and by-products.

In some embodiments, the products from the Diels-Alder condensation reactions may be subjected to an isomerization step. Suitable reaction catalysts to effect the isomerization reaction may include acid catalysts as described herein. The temperature at which the reaction may range from about 50° C. to about 400° C., preferably from about 120° C. to about 180° C. Suitable catalysts used for isomerization step include Lewis-Bronsted acid catalysts.

Suitable products from the Diels Alder condensation reaction, or the following isomerization step include, but not limited to:

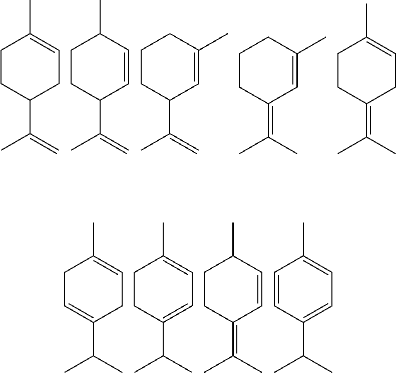

In various embodiments, terpene mixture containing limonene-carvestrene can be directly used as a “green” solvent alternative to toxic solvents such as halogenated solvents obtained from fossil fuel sources or a co-solvent having various applications such as in cleaning formulations, degreaser and as a lubricant in drilling application. Cleaning products for industrial and household applications represents the largest market for limonene and similar monoterpenes.

The isoprene and/or the terpenes made by the reactant mixture of the invention are also used to produce a tackifier that are substantially devoid of C5 or other bio-byproduct impurities. Thus, in another aspect, the invention provides a tackifier made from isoprene, terpenes or combinations thereof and a process for synthesizing a tackifier composition that are made from the reactant mixture of the invention. Tackifiers are generally low molecular weight compounds used in formulating adhesives to increase tack or the stickiness of the surface of the adhesive. Tackifiers tend to have glass transition and softening temperature above room temperature, providing them with suitable viscoelastic properties. Tackifying compositions can then be produced by oligomerization of the isoprene and/or the terpenes of the invention. The process of the invention is conducted such that a concentration of a sustainable content of the first compound or its precursor is greater than about 10% by weight, preferably greater than about 20% by weight, preferably greater than about 30% by weight, preferably greater than about 40% by weight, preferably greater than about 50% by weight, preferably greater than 60% by weight, preferably greater than about 70% by weight, preferably greater than about 80% by weight, preferably greater than about 90% by weight. This will result in a tackifying composition that has a substantially high sustainable content.

The process of the invention may also include a fractionating step to increase the purity of the monomer composition before polymerization. The process of the invention is also used to provide chain terminators such as methyl butenes e.g. 2-methyl-1-butene or 3-methyl-1-butene in the monomer composition. Chain terminators, as is well known in the art, are used to terminate growth of polymer chains thus limiting the polymer molecular weights within certain ranges. The nature of chain terminators depends on the type of polymerization and the conditions used for polymer synthesis. In the process of the invention used to produce tackifiers, mono-enes such as 2-methyl-1-butene, polar compounds such as ethanol, and the like can act as chain terminators. One skilled in the art will also understand that polymerization inhibitors at certain concentration levels can serve as chain terminators.

In some instances, the isoprene is subjected to further reactions, such as a dimerization step, to produce further compounds comprising at least one double bond. Exemplary compounds include limonene, carvestrene, dimethylcyclooctadienes, and the like, and combinations thereof. The monomer composition obtained from the process described herein may include at least one of 1-butene, 2-butene, isoprene, monoterpenes, sesquiterpenes, diterpenes, polyterpenes, acrolein, acrylic acid, hydroxymethyl furfural, dipentene, limonene, carvestrene, 4-ethenyl-1,4-dimethyl cyclohexene, terpinolenes, dimethyl cyclooctadienes, 4-ethenyl-1,4-dimethyl cyclohexene, dimethyl cyclooctadienes, 3-methyl-1,3-butadiene, α-terpinene, γ-terpinene, terpinolenes, isoterpinolene, isolimonene, isocarvestrene, p-cymene derived sesquiterpenes, p-cymene derived diterpenes, or combinations thereof. In some instances, the monomer composition comprises limonene at a concentration greater than about 10% by weight, preferably greater than about 20% by weight, preferably greater than about 30% by weight, preferably greater than about 40% by weight, preferably greater than about 50% by weight. In other instances, the monomer composition comprises carvestrene at a concentration greater than about 10% by weight, preferably greater than about 20% by weight, preferably greater than about 30% by weight, preferably greater than about 40% by weight, preferably greater than about 50% by weight. In further instances, the monomer composition comprises a mixture of limonene and carvestrene at a concentration greater than about 10% by weight, preferably greater than about 20% by weight, preferably greater than about 30% by weight, preferably greater than about 40% by weight, preferably greater than about 50% by weight, preferably greater than about 60% by weight, preferably greater than about 70% by weight, preferably greater than about 80% by weight, preferably greater than about 90% by weight. The limonenes and carvestrenes may then be oligomerized or polymerized to provide the tackifying compositions of the inventions.

As noted herein, polymerization may be effected through any known means in the art, such as free radical polymerization, ionic polymerization including cationic and anionic polymerization, ring-opening metathesis polymerization (ROMP), and the like. Depending on the choice of monomers, ratio of monomer to chain terminators, polymerization conditions, the chain length, molecular average, polydispersity index, such polymer properties can be suitably tailored. For example, increasing the concentration of carvestrene in the monomer composition will result in polymers having higher softening temperatures. One skilled in the art will also appreciate that the terms oligomer and oligomerization may also be used in place of polymer and polymerization respectively here.

In yet another aspect, the invention provides a process for synthesizing a polymer composition from derivatives of isoprene, and a polymer composition therefrom. Derivatives of isoprene produced from the process described here starting with the reactant mixture as described herein may include for example, dimethylcyclooctadienes (DMCOD).

Derivatization reactions may include, for example, Diels-Alder reactions, dimerization reactions, and the like, and combinations thereof. In some instances, the monomer composition comprises dimethyl cyclooctadienes, while in other instances, the monomer composition comprises isomers of dimethylvinylcyclohexene. The sustainable monomer composition comprises isoprene at a concentration greater than about 10% by weight, preferably greater than about 20% by weight, preferably greater than about 30% by weight, preferably greater than about 40% by weight, preferably greater than about 50% by weight, preferably greater than about 60% by weight, preferably greater than about 70% by weight, preferably greater than about 80% by weight, preferably greater than about 90% by weight.

By carefully controlling the relative concentrations of the chain terminators and the monomers, the resulting molecular weight distribution of the tackifier composition can be controlled. In some embodiments, the resulting tackifier composition comprises dimer molecules at a concentration less than about 10% by weight, preferably less than about 5% by weight, preferably less than about 3% by weight of dimer molecules.

The products from the Diels-Alder condensation reactions containing the double bonds can be hydrogenated to yield cycloalkane derivatives that are suitable as sustainable aviation fuels (SAFs). The hydrogenation reaction may be a complete or a partial hydrogenation reaction. Hydrogenation is effected using appropriate catalysts such as, for example, palladium, platinum, ruthenium, rhenium, nickel, copper, silver, or gold, supported on various supports such as carbon, silica, and/or alumina. The reactant mixtures and methods described herein would enable practical implementation of large scale production of not only sustainable aviation fuels but also various industrially useful compounds.

Suitable reaction conditions such as temperature, pressure, amount of catalysts, and the like, can be arrived at without undue experimentation by one of ordinary skill in the art. For example, the dehydration-decarboxylation reactions to convert mevalonolactone to isoprene may be performed at a temperature range of about 200-350° C. The Diels-Alder reaction may be conducted at a temperature of over about 150° C. The complete or partial hydrogenation may be conducted at temperature less than about 200° C. at hydrogen pressures of about 600-1200 psi. The time of reaction may range from about 1 hour to about 72 hours.

Thus, an exemplary specific reaction sequence to obtain a final product is:

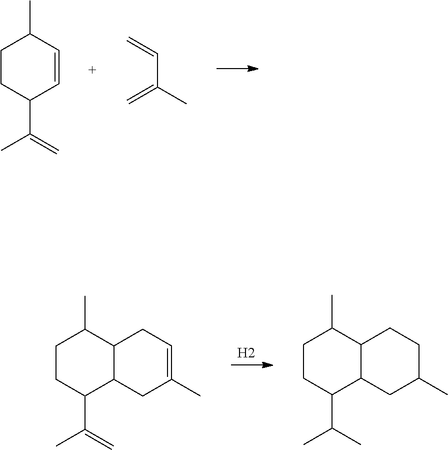

Exemplary non-limiting useful products resulting from the hydrogenation reaction include:

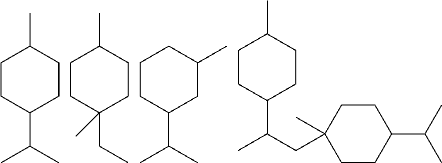

The reactant mixture and the corresponding processes involving the reactant mixture provide products that have very low impurities content, and in some instances, products that are generally devoid of impurities. Such impurities include, for example, sulfur, which are known to impart deleterious properties to the final product at high enough concentrations, and while they are tolerated at low concentrations, it is highly desirable to completely eliminate them from the final product. However, current manufacturing processes result in products having relatively high levels of sulfur and further reduction of sulfur typically requires additional steps thus increasing the costs. In some specific embodiments, the products of the invention have a sulfur content of less than 5 ppm.

The multiple reactions may be performed in the same reaction pot or may be conducted as separate reactions. One skilled in the art would understand that conducting the reaction in the same reaction pot would reduce further steps such as isolation of the intermediate products, thus economizing the process. Heat transfer fluids can advantageously be used for the additional reaction steps. It would also become obvious to one skilled in the art that the additional reaction steps may require additional catalysts and reagents.

One skilled in the art will also understand that additional steps may have to be performed in order to isolate and/or purify the products. These steps include, for example, distillation, chromatographic separation, crystallization, liquid-liquid extraction, liquid-solid extraction, and the like, and combinations thereof.

In some embodiments, the reactant mixture described herein is used in a process to produce compositions that are useful as, for example, flavorants, fragrances, fuels, fuel blends. As stated herein, the reactant mixture is capable of producing products with high specificity. In specific embodiments, the composition used for fuel blending is substantially free of C₇ hydrocarbons. It is generally known in the art that when starting materials from sustainable processes are used in conventional processes to produce compositions used for fuel blending, then a number of by-products that include undesirable C₇ hydrocarbons are also present in the final product. C7 hydrocarbons are generally undesirable in certain classes of fuels due to their vapor pressure, and hence it can be highly desirable to avoid them entirely. One skilled in the art would appreciate that if, for example, preparing an aviation fuel, it would be highly advantageous to avoid C7 hydrocarbons as a by-product of the reactions, thus eliminating the step of removing the C7 hydrocarbons from the final product entirely. The reaction mixture of the invention has been found to enable obtaining desired final products with a high level of selectivity with the minimum number of steps from a suitable starting material, thus ensuring the costs of production are kept at manageable levels.

There is much interest in the production of sustainable fuel compositions for use as replacements or partial replacements of fossil based fuel compositions. Many such compositions are known, as are processes for their preparation, however, these compositions and the processes for their preparation generally have undesirable features that detract from the value of their sustainability advantages. Such features can include, but are not restricted to, problems with undesirable minor components such as sulfur, undesirable physical properties such as an inappropriate freezing point or flash point or density or inappropriate boiling point curves or undesirable economic cost due to poor process yields or complex processing requirements.

In another aspect, the present invention generally relates to new sustainable fuel compositions and an innovative process for their preparation. The compositions have properties that make them particularly suitable for use as fuels, overcoming some of the aforementioned limitations of existing sustainable fuel compositions in certain applications.

Exemplary compounds that are useful in the composition for fuel blending including at least one of isopropyl-methylbenzene isomers, saturated and unsaturated dimethylcyclooctane isomers, isopropyl-methylcyclohexane isomers and 4-ethyl-1,4-dimethylcyclohexane, 4-ethyl-1,4-dimethyl cyclohexene isomers, and mixtures thereof. The compounds described herein may be present at a concentration of about 50% w/w, or preferably of about 60% w/w, or preferably of about 70% w/w, or preferably of about 80% w/w or preferably about 90% w/w.

In certain embodiments, the composition used for fuel blending comprises at least about 5 wt % aromatic compounds, or preferably at least about 10 wt % aromatic compounds, or preferably about 15 wt % aromatic compounds, or preferably at least about 20 wt % aromatic compounds, or preferably at least about 25 wt % aromatic compounds, or preferably at least about 30 wt % aromatic compounds. In other embodiments, the composition used for fuel blending comprises at least about 5 wt % alicyclic compounds, or preferably at least about 10 wt % alicyclic compounds, or preferably about 15 wt % alicyclic compounds, or preferably at least about 20 wt % alicyclic compounds, or preferably at least about 25 wt % alicyclic compounds, or preferably at least about 30 wt % alicyclic compounds.

The composition used for fuel blending may have suitable properties such as, for example, but not limited to, a density greater than 0.85 g/cm³ at 20° C., and a flash point greater than 38° C. Further, the composition may have a sulfur content less than about 5 ppm.

In other embodiments, the composition for fuel blending comprises C₁₀ hydrocarbons. The C₁₀ hydrocarbons may be present at a concentration greater than about 10% w/w, or preferably greater than about 20% w/w, or preferably greater than about 30% w/w, or preferably greater than about 40% w/w, greater than about 50% w/w, or preferably greater than about 60% w/w, or preferably greater than about 70% w/w, or preferably greater than about 80% w/w, or preferably greater than about 90% w/w. In specific embodiments, the C₁₀ hydrocarbon useful in the invention include isomers of cymene.

The composition for fuel blending may further comprise the heat transfer fluid. The presence of the heat transfer fluids in the final product will not affect the properties of the final product or may in certain circumstances enhance the properties of the final product.

In another embodiment, the invention provides fuel compositions comprising the composition used for fuel blending. The fuel may further comprise at least one of paraffins and/or isoparaffins. In specific embodiments, the fuel blend is used in a jet fuel composition. The reactant mixture and processes described herein are used to produce the compositions used for fuel blending that comprise a sustainable aromatic content less than about 17% w/w of the fuel blend, or preferably less than about 15% w/w of the fuel blend, or preferably less than about 13% w/w, or preferably less than about 11% w/w of the fuel blend, or preferably less than about 9% w/w of the fuel blend, or preferably less than about 7% w/w of the fuel blend, or preferably less than about 5% w/w of the fuel blend, or preferably less than about 3% w/w of the fuel blend.

There is much interest in the production of sustainable fuel compositions for use as replacements or partial replacements of fossil based aviation fuel compositions. Many such compositions are known, as are processes for their preparation, however, these compositions and the processes for their preparation generally have undesirable features that detract from the value of their sustainability advantages. Such features can include, but not restricted to, problems with undesirable minor components such as polyaromatic hydrocarbons whose combustion exacerbates problems with contrail formation or sulfur which is linked to corrosion issues and which is a precursor to sulfur oxide pollution. Other problematic features include undesirable physical properties such as an inappropriate freezing point or flash point or density or boiling point curves or undesirable economic cost due to poor process yields or complex processing requirements.

In another aspect, the present invention generally relates to new sustainable aviation fuel compositions useful as aviation fuel blending components or directly as a finished fuel where regulations permit. The compositions of the invention have properties that make them particularly suitable for use as fuels, overcoming some of the aforementioned limitations of existing sustainable aviation fuel compositions.

The sustainable aviation fuel composition comprises aliphatic and/or cycloaliphatic hydrocarbons and further comprises aromatic hydrocarbons, wherein the aromatic hydrocarbons are almost exclusively monocyclic aromatic hydrocarbons (MAHs) and very little to no polycyclic aromatic hydrocarbons (PAHs). The reactant mixture of the invention and the process of the invention involving starting materials obtained from sustainable sources enables production of the sustainable aviation fuel composition having very low concentrations of PAHs without compromising the concentrations of MAHs. In exemplary embodiments, the MAHs are present in a concentration ranging from about 1% by volume to about 25% by volume. In other exemplary embodiments, PAHs are present in a concentration ranging from about 0% by volume to about 5% by volume. In further exemplary embodiments, the concentration of the PAHs ranges from about 0.1% by volume to about 3% by volume, or preferably from about 0.01% by volume to about 1.5% by volume, or preferably from about 0.02% by volume to about 1.5% by volume, or preferably from about 0.03% by volume to about 1.5% by volume.

In specific embodiments, the MAHs comprise cymenes. Cymenes may be exclusively paracymene, or exclusively metacymene, or mixture of paracymene and metacymene. The concentration of cymenes may range from about 10% by weight to about 100% by weight of the total MAHs present in the sustainable aviation fuel of the invention.

The sustainable aviation fuel composition may further comprise heat transfer fluid.

The fuel blend composition can be characterized by a Carbon-12-Carbon-14 isotope ratio that indicates a renewable carbon content of more than about 10%, preferably more than about 20%, or preferably more than about 30%, or preferably more than about 40%, or preferably more than about 50%, or preferably more than about 60%, or preferably more than about 70%, or preferably more than about 80%, or preferably more than about 90%, or preferably more than about 99%. In other embodiments, the fuel blend composition comprises less than about 10%, or preferably less than about 12%, or preferably less than about 14%, or preferably less than about 16%, or preferably less than about 18%, or preferably less than about 20%, or preferably less than about 25% w/w fossil fuel aromatic hydrocarbons.

The sustainable aviation fuel composition or fuel blending composition has a freezing point that preferably is below minus 40° C.

In certain embodiments, the sustainable aviation fuel composition or fuel blending composition can have an unusually high volumetric energy content, wherein the composition has an energy content ranging from about 120000 BTU/US gallon to about 150000 BTU/US gallon.

The sustainable aviation fuel composition or sustainable fuel blend component is particularly advantageous wherein the sulfur content of the composition is less than about 1000 ppm, or preferably less than about 700 ppm, or preferably less than about 500 ppm, or preferably less than about 300 ppm, or preferably less than about 200 ppm, or preferably less than about 100 ppm, or preferably less than about 50 ppm, or preferably less than about 20 ppm, or preferably less than about 10 ppm.

As already noted herein, the presence of MAHs imparts useful properties such as fuel seal swell properties that enhances its use as jet fuel.

Thus, in another aspect, the invention provides a process for synthesizing a sustainable aviation fuel composition or sustainable fuel blending component. The process comprises:
a. providing a reaction mixture comprising renewable mevalonate;
b. converting the mevalonate in the reaction mixture to provide a first intermediate comprising isoprene;
c. reacting the isoprene in the first intermediate in the presence of a first heat transfer agent to provide a second intermediate comprising terpenes; and
d. reacting the second intermediate in the presence of a second heat transfer agent to provide the sustainable fuel composition.

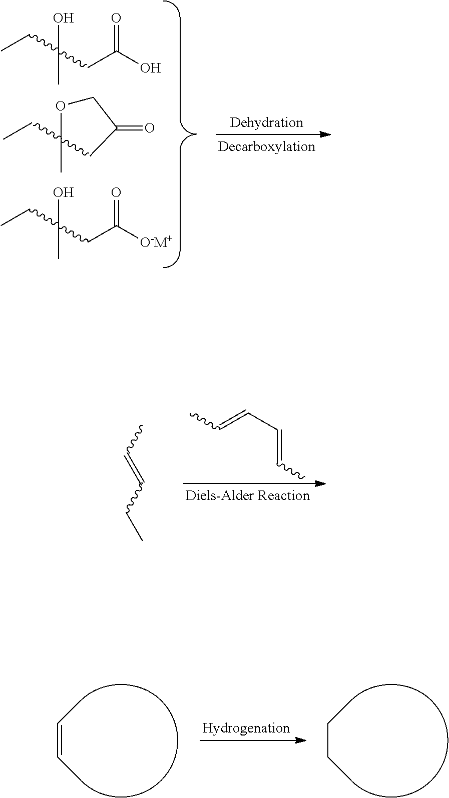

The sustainable aviation fuel composition made by the process described herein comprises MAHs at a concentration ranging from about 1% by volume to about 25% by volume, and PAHs at a concentration ranging from about 0% by volume to about 3% by volume.

In certain embodiments, the first and/or second heat transfer agent comprises an aliphatic component, while in other embodiments, the first and/or second heat transfer agent comprises a cycloaliphatic component. In specific embodiments, the first and/or second heat transfer agent comprises Hydrotreated Esters and Fatty Acids (HEFA), wherein the heat transfer agent may comprise a MAH component. In other specific embodiments the first and/or second heat transfer agent comprises paraffinic hydrocarbons derived from Fischer Tropsch technology (FT).

The first intermediate may additionally comprise butadiene. In some alternate embodiments, one or more C₄ to C₁₂ olefins may be introduced before step b. Thus, the process of the invention allows for greater flexibility in the final product chemical structure, while still maintaining specificity and considerably reducing the impurities in the final product.

Also, as noted herein, the process of the invention also includes oligomerization and/or polymerization of monomers containing at least one double bond made by the process described herein. The monomers are substantially devoid of the impurities typically associated with the monomers obtained from petroleum based sources or other sustainable resources/processes. The monomers may further be tailored to include specific concentrations of chain terminators. This can be achieved by suitable fractionation steps of the products such as isoprene from the process described herein. Fractionation is well known in the art and may include, for example chromatographic separation, distillation, and the like, and combinations thereof.

The process may further comprise hydrogenating at least a portion of the MAHs to produce a sustainable fuel composition comprising MAHs and cycloaliphatic hydrocarbons.

At least one of the reaction mixture, the first intermediate, and the second intermediate comprises a heat transfer fluid. Heat transfer fluids may be advantageously used as a reaction medium to conduct the reactions stated herein to obtain the intermediates and products. One skilled in the art would understand that the cycloaliphatic content in the sustainable fuel composition may be controlled by effecting suitable separation methods known in the art. In some embodiments, the sustainable aviation fuel composition obtained from the process comprises the heat transfer fluid. It would be obvious that this would simplify the process steps and thus, can render the process more economical.

In other embodiments, the process of the invention involves starting with renewable 1,3-butadiene in the first intermediate mixture. This would then be used to obtain the second intermediate comprising cyclooctadiene and vinylcyclohexene, which is then used to obtain the sustainable fuel composition.

There has been a great deal of attention given to the invention of sustainable fuels, produced using renewable feedstocks. The majority of this work has tended to focus on alternatives to fossil road transport fuels and more recently on alternatives to fossil aviation fuel compositions. Much less attention has been given to inventing sustainable alternatives to fossil fuels for rocket engines.

Rocket fuels have particular special requirements. For example, the weight of fuel required is an important parameter and so the lower heating value of the fuel is an important consideration. An above average lower heating value is an attractive feature for hydrocarbon rocket fuels such as Rocket Propellant-1 (RP-1). Very low sulfur content is important as sulfur damages hardware at high temperatures, causes pollution and catalyzes polymerization. Also, it is preferable that rocket fuels have a low olefins and aromatics content because these tend to polymerize in regenerative cooling channels, and can also gum up hardware in rockets when stored for long periods. More branched-chain alkanes in rocket fuels would provide advantages as this improves thermal stability during regenerative cooling. Further, a relatively narrow range of molecular weights is beneficial in order to keep the lubricating properties consistent and to keep the fuel composition from changing due to separation or differential evaporation

In yet another aspect, the present invention provides a sustainable fuel composition suitable for use as a rocket propellant, akin to RP1 type fuels but with an alicyclic hydrocarbon content delivering good energy content and particularly low sulfur levels and low temperature properties. Specific embodiments of the invention provide usefully narrow spread of molecular weight in the hydrocarbon composition of the sustainable fuel.

The rocket fuel composition comprises alicyclic hydrocarbons and optionally aromatic hydrocarbons. The rocket fuel composition comprises renewable composition that comprises alicyclic hydrocarbons that comprises alkyl substituted cyclohexanes and/or alkyl substituted cyclooctanes. The total renewable content is at least 5% w/w of the composition, preferably 10% w/w of the composition, more preferably 15% w/w of the composition, more preferably 20% w/w of the composition, more preferably 25% w/w of the composition, more preferably 30% w/w of the composition.

The alicyclic hydrocarbon content of the rocket fuel composition is greater than 5% w/w of the composition, preferably 10% w/w of the composition, preferably 15% w/w of the composition, more preferably 20% w/w of the composition, more preferably 25% w/w of the composition, more preferably 30% w/w of the composition. In some specific embodiments, the aliphatic hydrocarbon is comprised of substantially 10 carbon hydrocarbon molecules. The concentration of the 10 carbon hydrocarbon is greater than about 10% w/w of the composition, preferably greater than about 20% w/w of the composition, preferably greater than about 30% w/w of the composition, more preferably greater than about 40% w/w composition, more preferably greater than 50% w/w of the composition, more preferably greater than about 60% w/w of the composition, more preferably greater than about 70% w/w of the composition.

The rocket fuel composition comprises polyaromatic aromatic hydrocarbons (PAHs) at a concentration less than about 3% w/w of the composition, preferably less than about 2% w/w of the composition, more preferably less than about 1% w/w of the composition.

The aromatic hydrocarbon in the rocket fuel composition is substantially comprised of cymene isomers.

As noted herein, the composition made by the process of the invention results in products generally devoid of impurities. Thus, the invention provides the rocket fuel composition that has a sulfur content less than about 30 ppm, or preferably less than about 25 ppm, or preferably less than about 20 ppm, or preferably less than about 15 ppm, or preferably less than about 10 ppm, or preferably less than about 5 ppm, or preferably less than about 1 ppm, or preferably less than 0.1 ppm. Further, the control and specificity of the process allows for the rocket fuel composition to have PAHs less than about 3% w/w, or preferably less than about 2% w/w, or preferably less than about 1% w/w.

The rocket fuel composition of the invention has certain properties that renders it useful for the application, such as having a freezing point lower than about −40° C. Further, the rocket fuel composition of the invention has a heat content of at least about at least 120000 BTU/US gallon, preferably at least about 121000 BTU/US gallon, preferably at least about 122000 BTU/US gallon, preferably at least about 123000 BTU/US gallon, preferably at least about 124000 BTU/US gallon, more preferably at least about 125000 BTU/US gallon at 15° C.

In yet another aspect the invention relates to the production of monomer compositions suitable for oligomerisation or polymerisation to provide renewable oligomers for uses such as tackifier products or renewable polymers. Such polymers can be renewable homopolymers or can be for example renewable block copolymers. The monomer compositions of the invention have certain additional properties such as the substantial absence of chain terminating contaminants hence rendering the composition particularly suitable for polymer formation. Limonene is a component often used in such compositions. We have shown that by synthesising C10 compounds from isoprene using a heat transfer agent in the reaction mixture, we achieve terpene blends which include limonene but also include carvestrene, which show an improved polymer yield with properties suitable for use as a tackifier.

It is known in the art that fermentation derived isoprene can be polymerised. For example in U.S. Pat. No. 8,901,262 B2 Example 18, isoprene derived from glucose was polymerised using the techniques of solution polymerisation and emulsion polymerisation. Polyisoprene was produced using a neodymium catalyst. This polymer had a weight average molecular weight of 935,000. Using a titanium catalyst a polyisoprene polymer with a weight average molecular weight of 221,000 was produced. The absence or substantial absence of chain terminating compounds is an important factor in successfully achieving desired polymer molecular weights.

### EXAMPLES

### Example 1

Plasmid and E. coli Strain Construction

Genetic segments encoding mvaE (acetyl-CoA acetyltransferase/HMG-COA reductase, GenBank No. AAG02438) and mvaS (HMG-COA synthase, GenBank No. AAG02438) in Enterococcus faecalis V583 were amplified from its genomic DNA. These segments were inserted into a vector (with pBR322 origin back bone, Ampicillin marker, lacIq, rrnB transcription termination sequences) under the control of an IPTG inducible Trc promoter-lac operator to obtain plasmid pSEI.

Chemically competent E. coli cells of XL-IBlue strain (endA1 gyrA96(nal^{k}) thi-1 recA1 re1A1 lac glnV44F[::Tn10 proAB⁺ lacIq Δ(lacZ)M15] hsdR17(r_{K}_{^{−}}m_{K}_{^{−}})) were transformed with plasmid pSEI using the procedures outlined in Sambrook-Maniatis (Green, M. R.; Sambrook, J. Ed. Molecular Cloning: A Laboratory Manual, Fourth Edition, 2002), the entire disclosure of which is incorporated herein by reference in its entirety, to obtain strain E. coli-strain-SE1.

### Example 2

### Mevalonolactone Production

E-coli SE1 strain was propagated in LB medium supplemented with 100 μg/L ampicillin in 250 mL of media in a 1 L flask, which is incubated at 37° C., in an orbital shaker at 220 rpm for 10 h reaching an OD600 of 3. This was used as an inoculum for the production of an Infors 51t bioreactor. 1.75 liter of production media containing 15 g/L glucose, 7 g/L KH₂PO₄, 1 g/L NH₄Cl, 5 g/L yeast extract, 1 g/L citric acid, 2 g/L MgSO₄, 200 mg/L FeSO₄, 10 mg/L thiamine·HCl, and 10 mg MnSO₄ was combined with 250 mL of inoculum in the bioreactor. The pH of the system was maintained at 7 using a 20% NH₄OH solution and the temperature was maintained at 32° C. Air was sparged at 2 liters per minute (LPM) and the agitation was maintained at 700 rpm. 10 hours after inoculation, 1 mL of 1M IPTG was added to the bioreactor followed by the addition of anti-foam, if needed. Glucose concentration was maintained at 10 g/L by the addition of 600 g/L glucose solution to the bioreactor at 2 h intervals. The bioreactor was stopped after 2 days. Cells were then separated from the broth using a 0.45 microfilter to obtain the clear broth. Mevalonolactone concentration was found to be nearly 80 g/L at the end of the fermentation procedures.

### Example 3

### Conversion of Mevalonolactone to Isoprene Using Solid Acid Catalysts

After separation of the cells and biological debris using tangential flow filtration, the MVL mother liquor obtained from Example 2, was concentrated by the removal of water and other impurities via evaporation and filtration to obtain high purity MVL. Reactions were performed in 300 mL PARR reactors using mevalonolactone (MVL) as a feed, a heat transfer liquid such as Duratherm 600 as bulk media suspending 10 g of solid silica catalyst. In an alternate configuration the solid catalyst was loaded in a basket submerged in the heat transfer fluid bulk media, resulting in similar outcomes. The reaction temperature was monitored with a thermocouple inserted into the reaction media. In detail, the tests were performed with 100 g of heat transfer liquid and 60-100% of purified mevalonolactone solution in water was added into the reaction conditions using an HPLC pump. The reaction mixture was heated to 250-350° C. at atmospheric pressure while being stirred at 800-1300 RPM. The reactor outlet was connected to a Pot 1 and Pot 2 which was connected in series. Pot 1 was kept at a temperature of 30-50 C while Pot 2 was kept at lower temperatures (˜−40-60 C) using dry ice/ethanol mixture. The Parr reactor was first flushed with nitrogen a couple of times and pressurized with nitrogen to check for any possible leaking before starting the reaction. After pressure testing, the temperature was slowly increased to 280° C., with continuous stirring at ˜1000 RPM and with a nitrogen flow of 15-20 sccm. After reaching the set temperature, 70% solution of MVL was fed to the PARR reactor using an HPLC pump at a flow rate of 0.2-3 mL/min for 5-300 hours. After finishing the reaction, the reactor was flushed further with nitrogen for another 30 minutes to release any trapped Isoprene to Pot 2. Heavies, if any, produced during the reaction and water produced during the reaction were condensed in Pot 1, while Isoprene was collected in Pot 2 with high purity. Samples were taken at different time intervals to check the progress of the reaction and after the reaction was completed, the samples were analyzed using gas chromatography (GC-FID) and high-performance liquid chromatography (HPLC). The liquid products were further confirmed using standard compounds and were also analyzed on GC-MS to identify unknown compounds. Known massed solutions of these compounds were analyzed in GC-FID to create standards and calibrations. Experimental solutions were then compared with these calibration standards to quantify concentrations of the known compound. Reactions were also conducted in a flow reactor which showed similar results and long-term stability in the presence of a mixture of MVL and heat transfer fluids.

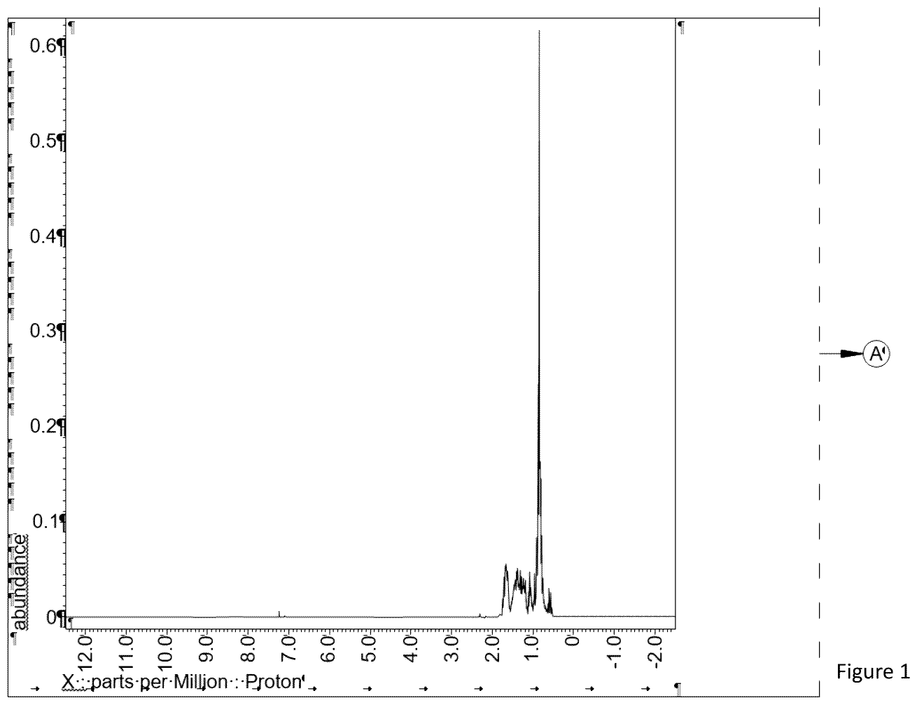

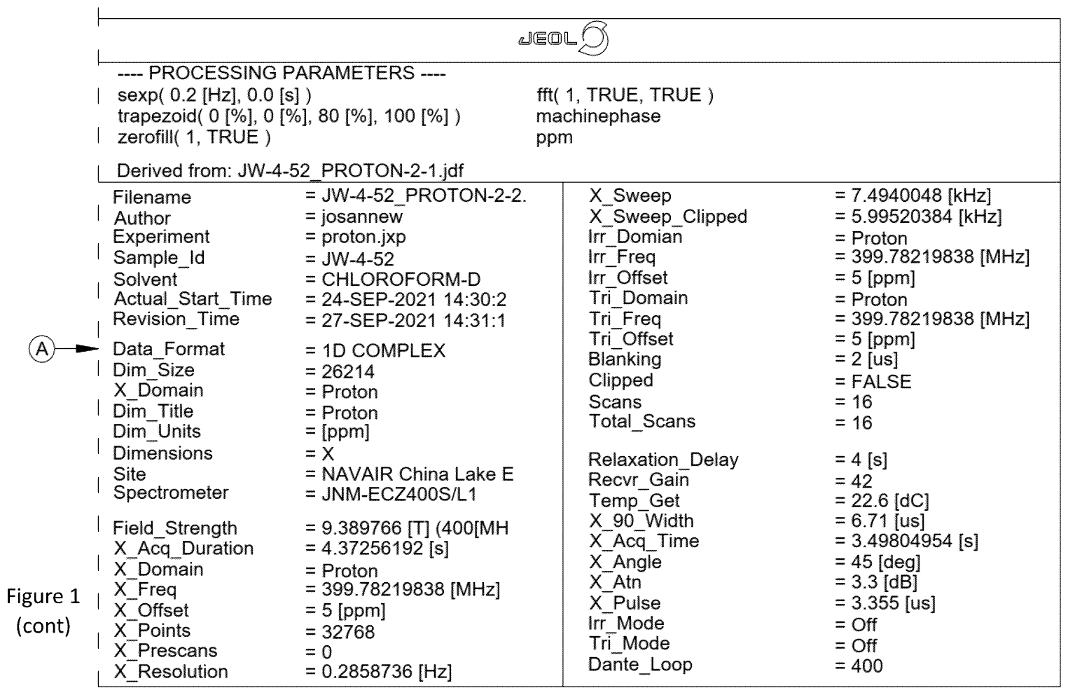

Analysis of the liquid products of the reaction, using metal oxide as catalyst, showed no presence of the mevalonolactone feed in the product in temperatures higher than 250° C., indicating that full conversion of mevalonolactone was achieved under the conditions evaluated. The composition of the liquid products as analysed by GC-FID is shown in Table 1. The main product obtained in Pot 1 was water followed by dMVL and small amounts (<2%) of organics like monoterpenes such as limonene and carvestrene, while isoprene was noted in Pot 2 from 250° C. onwards with purity >98% (FIG. 1). Thus, at temperatures in excess of 250° C., simultaneous dehydration and decarboxylation reactions of MVL occurred to produce isoprene with high purity that was collected in Pot 2. Minor impurities like methyl butenes (2-methyl 2-butene, 2-methyl 1-butene, etc) and pentadienes were also detected along with isoprene at below 2% level in Pot 2. Some of these impurities are also found in petro-based isoprene but in this example these are of bio-origin. Furthermore, other impurities like cyclopentadiene present in petro-based isoprene is absent, i.e. this isoprene composition is distinct from conventional petro-isoprene. Surprisingly, bio-byproduct impurities common in fermentation derived isoprene like methyl butanols and dimethyl disulfide are not detected in present bio-isoprene. It can be stated that there is a significant difference in composition between the present catalytically derived bio-isoprene and conventional isoprene derived from petroleum as well as bio-isoprene derived from fermentation.

Increasing the reaction temperature marginally changed the isoprene yield above 280° C. while terpenes as well as high molecular weight products were noted at higher reaction temperatures (350° C.).

TABLE 1
Analysis of the liquid products by GC-FID and GC-MS
obtained after the dehydration-decarboxylation reaction
of MVL at different reaction temperatures.

| Condition | 1 | 2 | 3 | 4 | 5 |
|---|---|---|---|---|---|
| Temperature, ° C. | 250 | 280 | 300 | 320 | 350 |
| Time, h | 5 | 5 | 5 | 5 | 5 |
| Main reactant | MVL | MVL | MVL | MVL | MVL |
| GC analysis, % | | | | | |
| Mevalonolactone | 10 | 1 | 0 | 0 | 0 |
| dMVL | 3 | 2 | 0 | 0 | 0 |
| Limonene/Carvestre | 2 | 2 | 1 | 6 | 14 |
| ne | | | | | |
| Isoprene | 80 | 94 | 97 | 92 | 82 |
| Others | 5 | 1 | 2 | 2 | 4 |

The reaction was repeated at a temperature of 290 C but using only half as much Duratherm 600 heat transfer fluid (50 ml instead of 100 ml). The conversion of mevalonolactone remained over 99% but the yield of isoprene fell marginally from 95% to 92%, with remaining being dMVL, which can be recycled to produce isoprene via decarboxylation.

Separate reactions using 100 ml of p-xylene and 100 ml of tetradecane as the heat transfer fluid were also conducted. Equipment limitations obliged the xylene case to be run at 120 C and the tetradecane case to be run at 220 C. Mevalonate conversion was therefore lower at 29.5% when xylene was used as the heat transfer fluid and 95% when tetradecane was used as the heat transfer fluid. Isoprene yield was 8.5% in the xylene case and 48% in the tetradecane case while the remaining was the intermediate dehydromevalanolactone (dMVL), which can be recycled to produce more isoprene. Neither of these cases produced heavy products (e.g. terpenes) and no coke formation was observed, unlike the case where a heat transfer fluid was not used.

### Example 4

### Effect of Catalyst on Conversion of Mevalonolactone and Product Composition

For the below tests, reactions were performed using 5-20 wt. % of the catalysts in 300 mL Parr reactors using mevalonolactone as a feed and using 100 g of heat transfer liquid as solvent at 280° C. and at atmospheric pressure while being stirred at 1000 RPM. After 5h of the reaction, the liquid samples were centrifuged and analyzed using gas chromatography (GC-FID) and high-performance liquid chromatography (HPLC). The catalysts used for these reactions included titanium oxide, zirconium oxide, aluminium oxide, niobium oxide, niobium phosphate, aluminum trichloride, and silica-alumina. All catalysts showed isoprene as the main dehydrated product and among all the catalysts screened silicon oxide showed the highest yield of isoprene. The results are shown in the following Table 2.

TABLE 2
Analysis of liquid products obtained for Mevalonolactone
using different acid catalysts

| | Batch time, h | | | | |
|---|---|---|---|---|---|
| | 5 | 5 | 5 | 5 | 5 |
| | GC Analysis, % | | | | |
| Catalysts | MVL | dMVL | Isoprene | Terpenes | Others |
| TiO₂ | 2 | 7 | 83 | 5 | 3 |
| ZrO₂ | 1 | 5 | 88 | 4 | 2 |
| SiO₂ | 0 | 0 | 97 | 1 | 2 |
| Al₂O₃ | 0 | 8 | 75 | 15 | 2 |
| Nb₂O₅ | 5 | 10 | 80 | 0 | 5 |
| SiO₂—Al₂O₃ | 0 | 3 | 67 | 10 | 15 |

### Example 5

The liquid analysis from example 4 shows that for a range of catalysts in the presence of a heat transfer fluid, isoprene yields of over 65% were achieved via the decarboxylation of bioderived mevalonolactone, with a reaction time of 5 hours. No coking of the catalyst occurred.

It has been previously shown (ACS Catalysis, Vol 10/Issue 16, Research Article, Jul. 31, 2020: Efficient Route for the Production of Isoprene via Decarboxylation of Bioderived Mevalonolactone) that coke formation is a major selectivity loss in the decarboxylation of mevalonolactone to isoprene when a heat transfer agent is not used in the reaction system.

For example, FIG. 12 of that publication shows that over 2 hours of running the reaction over a silica alumina catalyst, more than 20% of the mevalonate feed had converted to coke. After 4 hours, more than 30% of the feed was being converted to coke.

This example thus demonstrates the substantial improvement in product yield and the major reduction in problematic coke formation which results from the use of the heat transfer agent in the reaction system,

### Example 6

A flow reactor of volume 140 ml was charged with a silica-alumina catalyst. The reactor was heated to 350 C and a feed comprising 70% by weight mevalonolactone and 30% by weight of water was fed to the reactor at a space velocity of 1 per hour. Over a period of 48 hours the reactor became plugged with coke and the reaction was stopped. The reactor was allowed to cool and the catalyst was drilled out of the reactor.

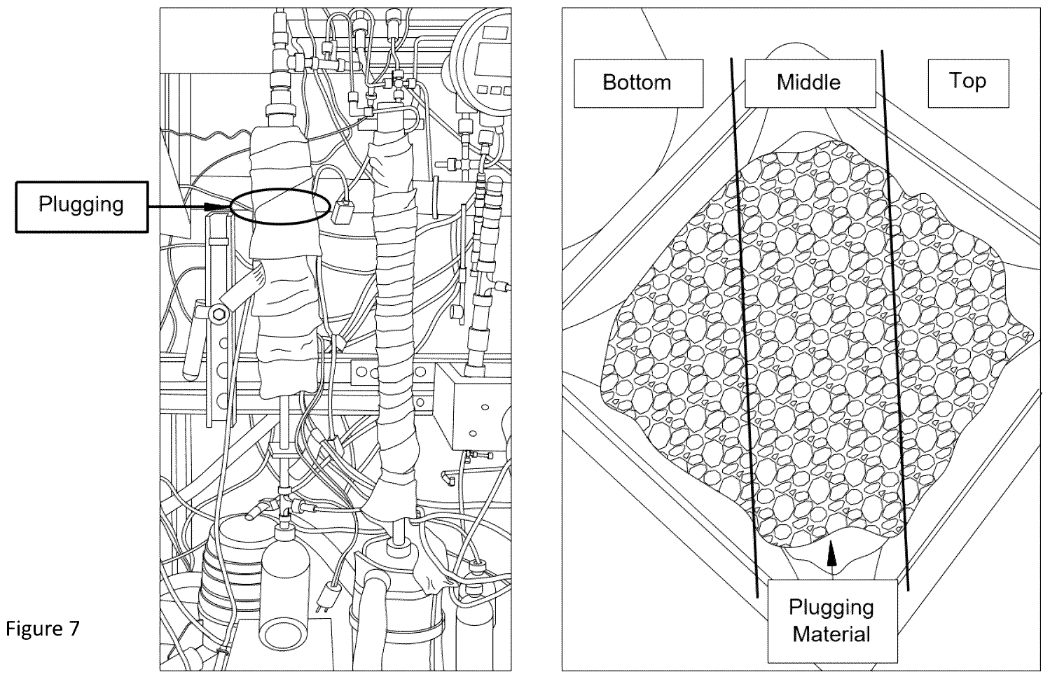

FIG. 7 shows the catalyst after removal. The discoloration due to coke formation can be seen.

### Example 7

### Selective Dimerization/Cycloaddition Reaction of Isoprene to Monoterpenes

Isoprene collected from the first step of the MVL dehydration-decarboxylation reaction is used in the synthesis of monoterpenes by a Diels-Alder cycloaddition reaction. Reactions were performed in 300 mL Parr reactors using isoprene as a feed and a heat transfer liquid as solvent and the reaction temperature was monitored with a thermocouple inserted into the reaction media. The tests were performed with 100 g of heat transfer liquid Duratherm 600 and an equivalent volume of isoprene in the absence of catalysts. The reaction was conducted in two different ways (a) Isoprene was added to the PARR reactor containing heat transfer fluid, using an HPLC pump at an isoprene flow rate of 1-3 ml/min and (b) The required amount of isoprene and the heat transfer liquid was mixed in the PARR reactor before it was heated up to the reaction temperature. Irrespective of the mode of isoprene addition, the PARR reactor was flushed multiple times with nitrogen before it was heated up to 160-250° C. while being stirred at 1000 RPM. Samples were taken at different time intervals to check the progress of the reaction and after the reaction was completed, the reactor was allowed to cool to room temperature, depressurized, and the liquid samples were centrifuged and analyzed using gas chromatography (GC-FID). The liquid products were further confirmed using standard compounds and were also analyzed on GC-MS to identify unknown compounds. Among the terpene products, limonene and carvestrene, account for 60-80% of the total terpene composition followed by 4-ethenyl-1,4-dimethyl cyclohexene and cyclooctadienes/terpinolenes (Table 3).

TABLE 3
Analysis of liquid products by GC-FID and GC-MS
obtained under different reaction temperatures.

| | Reaction time, h | | | | |
|---|---|---|---|---|---|
| | 5 | 5 | 5 | 5 | 5 |
| | T, ° C. | | | | |
| GC analysis, % | 160 | 180 | 200 | 220 | 250 |
| Isoprene | 20 | 5 | 0 | 0 | 0 |
| Limonene/Carvestrene | 63 | 71 | 76 | 78 | 70 |
| 4-ethenyl-1,4- | 12 | 15 | 16 | 15 | 14 |
| dimethyl cyclohexene | | | | | |
| Cyclooctadienes | 3 | 5 | 5 | 2 | 4 |
| Terpinolenes | 1 | 2 | 3 | 5 | 5 |
| Others | 0 | 0 | 0 | 0 | 7 |

### Example 8

### Cycloaddition/Dimerization of Isoprene to Monoterpenes in Presence of Catalysts

Reactions were again performed in 300 mL Parr reactors using isoprene as a feed and a heat transfer liquid as solvent in the presence and absence of various acid catalysts. The tests were performed with 100 g of heat transfer liquid and an equal volume of isoprene in the presence of selected catalysts including silicon dioxide, aluminum oxide, silica-alumina and titania. The reaction mixture was heated to 220° C. while being stirred at 1000 RPM. After completion of the reaction, the reactor was allowed to cool at room temperature, depressurized, and the liquid samples were centrifuged and analyzed using gas chromatography (GC-FID) and high-performance liquid chromatography (HPLC). Results are shown in Table 4.

TABLE 4
Analysis of liquid products by GC-FID and GC-MS obtained during
the conversion of isoprene in presence of different catalysts.

| | Reaction time, h | | | | |
|---|---|---|---|---|---|
| | 5 | 5 | 5 | 5 | 5 |
| | Catalyst | | | | |
| GC analysis, % | None | Silica | Alumina | Titania | Si—Al |
| Isoprene | 0 | 0 | 0 | 0 | 0 |
| Limonene/Carvestrene | 78 | 77 | 70 | 75 | 65 |
| 4-ethenyl-1,4- | 15 | 16 | 15 | 15 | 15 |
| dimethyl cyclohexene | | | | | |
| Cyclooctadienes | 2 | 4 | 2 | 2 | 3 |
| Terpinolenes | 5 | 3 | 8 | 8 | 10 |
| Others | 0 | 0 | 5 | 0 | 7 |

### Example 9

Dehydration-Decarboxylation of Anhydromevalonate and of Mevalonate, Using a Molten Salt Heat Transfer Fluid.

Reactions were performed in 300 mL Parr reactors using either anhydromevalonate (aMVL) or a 70% w/w solution of mevalonate (MVL) in water as a feed and Dynalene MS-2 molten salt system as the heat transfer liquid in the presence and absence of a silica catalyst. The tests were performed with 100 g of the heat transfer liquid and an equal volume of the aMVL or MVL.

The reaction conditions are shown in table 4. After reaction the contents were cooled, depressurized and centrifuged and analyzed using gas chromatography (GC-FID). Results are shown in Table 5. A comparison reaction is also shown where an organic liquid high temperature heat transfer fluid replaced the molten salt heat transfer fluid

TABLE 5
Analysis of the liquid products after the dehydration-decarboxylation
reaction of MVL and aMVL in presence of molten salts (MS) and
its comparison with heat transfer solvent Duratherm 600(HTS).

| Condition | 1 | 2 | 3 | 4 | 5 |
|---|---|---|---|---|---|
| Temperature, ° C. | 290 | 290 | 290 | 290 | 290 |
| SiO₂ Catalyst Used, g | 20 | — | 20 | — | 20 |
| Time/ h | 4 | 4 | 4 | 4 | 4 |
| Pressure/ atm | 2.5 | 2.5 | 2.5 | 2.5 | 2.5 |
| Main reactant | aMVL | aMVL | MVL | MVL | MVL |
| Feed Concentration/% | 100 | 100 | 70 | 70 | 70 |
| (balance is water) | | | | | |
| Heat Transfer Fluid Used | MS | MS | MS | MS | HTS |
| GC analysis, % | | | | | |
| Mevalonolactone | 0 | 0 | 1 | 2 | 0.3 |
| dMVL | 6 | 5 | 5 | 7 | 3 |
| Terpenes | 0 | 1 | 1 | 1 | 0 |
| Isoprene | 48 | 40 | 58 | 42 | 95 |

It can be seen that the molten salt heat transfer fluid successfully allowed the production of isoprene with minimal generation of heavy products. Only 1% of terpenes were generated and no coking. The organic liquid heat transfer fluid does however achieve a higher isoprene yield under these conditions again, with no coking.

### Example 10

Isomerization-Dimerization Reactions of Monoterpenes Produced from Mevalonolactone

For the below tests, 1 g of C10 monoterpenes such as limonene-carvestrene (LC) admixture was treated with 100 mg of solid acid catalysts under neat conditions in 12 mL Q-tube reactors. The reaction mixture was heated to 60-150° C. at atmospheric pressure in a pre-heated metal block while being stirred at 800 RPM via a magnetic stirrer. The mixture was held at semi-batch reaction conditions for 5 hours. After 5 hours, the mixture was cooled to room temperature and the liquid samples were centrifuged and were analyzed on gas chromatography (GC-FID) and high-performance liquid chromatography (HPLC). The liquid products were further confirmed using standard compounds and were also analyzed on GC-MS to identify unknown compounds. Experimental solutions were then compared with these calibration standards to quantify concentrations of the known compound. Various Lewis-Brønsted acid catalysts were used for the isomerization reaction. Among all the catalysts screened, Amberlyst-15 displayed ˜85% selectivity towards terpinolenes at 100° C./5h. The results are shown in table 6.

TABLE 6
Analysis of liquid products by GC-FID and GC-MS obtained in the isomerization-
dimerization reaction of C10 monoterpenes using different solid acid catalysts

| | Batch time, h | | | | |
|---|---|---|---|---|---|
| | 5 | 5 | 5 | 5 | 5 |
| | T, ° C. | | | | |
| | 100 | 100 | 100 | 100 | 100 |
| | GC Analysis, % | | | | |
| Catalysts | Terpinolene | Terpinene | Limonene/Carvestrene | Diterpenes | Others |
| Amberlyst-15 | 85 | 9 | 1 | 3 | 2 |
| Amberlyst-45 | 47 | 23 | 4 | 18 | 8 |
| Nafion NR50 | 37 | 20 | 5 | 16 | 22 |
| SiO₂—SO₃H | 24 | 15 | 7 | 11 | 43 |
| TiO₂ | 30 | 35 | 20 | 5 | 10 |
| Montmorillonite | 40 | 30 | 15 | 5 | 10 |
| Zeolite | 25 | 20 | 5 | 10 | 40 |
| SiO₂—Al₂O₃ | 20 | 25 | 8 | 12 | 35 |

Since Amberlyst-15 (A-15) showed high selectivity towards terpinolenes, effect of reaction temperature was evaluated further. For the below tests, 1 g of the monoterpene mixture was treated with 100 mg of A-15 under neat conditions in 12 mL Q-tube reactors. The reaction mixture was heated to 60-110° C. for 5 hours at atmospheric pressure in a pre-heated metal block while being stirred at 800 RPM via a magnetic stirrer. After 5 hours, the reaction mixture was cooled to room temperature and the liquid samples were centrifuged and analyzed using gas chromatography (GC-FID). The liquid products were further confirmed using standard compounds and were also analyzed on GC-MS to identify unknown compounds. As shown in Table 7, increasing the reaction temperature improved the overall selectivity towards terpinolenes and at 110° C., terpinolenes are produced in more than 95% selectivity. Meanwhile, at reaction temperatures of 50° C., dimerized C20 diterpene compounds are noted with nearly 35% selectivity over Amberlyst-15 catalyst. Thus, reaction temperatures of 110° C. is optimal for the high yield synthesis of terpinolenes while at lower reaction temperatures of 50-60° C., C20 diterpenes are produced using Brønsted acidic Amberlyst-15 catalysts.

TABLE 7
Analysis of liquid products by GC-FID and GC-MS obtained
in the isomerization-dimerization reaction of C10
monoterpenes under different reaction temperatures

| | Batch time, h | | | | |
|---|---|---|---|---|---|
| | 5 | 5 | 5 | 5 | 5 |
| | GC Analysis, % | | | | |
| | | | Limonene/ | | |
| T, ° C. | Terpinolene | Terpinene | Carvesterene | Diterpenes | Others |
| 50 | 37 | 10 | 12 | 33 | 8 |
| 60 | 43 | 11 | 2 | 35 | 9 |
| 80 | 64 | 17 | 1 | 15 | 3 |
| 100 | 85 | 9 | 1 | 3 | 2 |
| 110 | 96 | 2 | 0 | 0 | 2 |

The results clearly indicate that the synthesis of the target compounds are at least comparable to that reported in U.S. Pat. No. 9,682,897B1 at lower temperatures, and with increasing temperatures, the method of the invention surprisingly shows near exclusive selectivity towards the compound of interest. This level of selectivity towards a target compound with such a minimum number of steps involved in the conversion from a starting material from renewable resource has not been shown before.

### Example 11

Synthesis of C15 Sesquiterpenes from Mevalonolactone

For the below tests, 1 g of limonene or limonene-carvestrene (LC) terpene mixture was treated with 1 ml of isoprene using 100-200 mg of solid acid catalysts under neat conditions in metal Q tube reactors. The reaction mixture was heated to 120-180° C. for 5 hours at atmospheric pressure in a pre-heated oil bath while being stirred at 800 RPM via a magnetic stirrer. After 5 hours, the reaction mixture was cooled to room temperature and the liquid samples were centrifuged and analyzed on gas chromatography (GC-FID). The catalysts used for these reactions include solid Lewis-Brønsted acid catalysts such as Montmorillonite K10, titanium oxide, aluminium oxide, Nafion NR50, and a silica supported sulphonic acid catalyst. Results shown in table 8, suggest that the acid catalysts performed the selective isomerization reaction of LC mixture, and produced the C15 sesquiterpenes by the Diels-Alder reaction between the LC isomer and isoprene. For instance, reaction between limonene-carvestrene (LC) mixture and isoprene using Lewis acidic Montmorillonite catalysts produced C15 sesquiterpenes with nearly 40% selectivity at 150° C./5h. However, in the absence of catalysts, sesquiterpene formation was not observed suggesting that the isomerization reaction is a key step for Diels-Alder reaction with isoprene to overcome steric hindrance.

TABLE 8
Analysis of liquid products by GC-FID and GC-MS obtained
in the isomerization and Diels-Alder reaction of C10
monoterpenes with Isoprene using solid acid catalysts

| Condition | | | | | |
|---|---|---|---|---|---|
| T, ° C. | 150 | 150 | 150 | 150 | 150 |
| Catalysts | Mont. K10 | SiO₂—SO₃H | Nafion | Al₂O₃ | TiO₂ |
| | | | NR50 | | |
| GC analysis, % | | | | | |
| LC | 0 | 0 | 0 | 5 | 10 |
| LC Isomer | 49 | 60 | 72 | 75 | 70 |
| C15 Sesquiterpene | 43 | 25 | 18 | 20 | 15 |
| Others | 8 | 15 | 10 | 0 | 5 |

### Example 12

### Reusability of the Heat Transfer Liquid in the Cycloaddition Reaction of Isoprene

The heat transfer liquid used in the cycloaddition reaction of Isoprene to monoterpenes was tested in additional catalytic runs for its stability and reusability. For that, after reaction, monoterpenes were separated from the solution containing the heat transfer liquid and dissolved monoterpenes separated by a controlled distillation. The heat transfer liquid was collected after the distillation and was then used for additional Isoprene to monoterpenes runs. As shown in Table 9, the heat transfer fluid can be reused without much difference in the total yield of the products confirming the stability of the solvent and for further scale up of the process.

TABLE 9
Analysis of liquid products by GC-FID and GC-MS
obtained during the reuse of heat transfer fluid.

| Reaction time, h | 5 | 5 | 5 |
|---|---|---|---|
| T, ° C. | 200 | 200 | 200 |
| # of Cycles | 1 | 2 | 3 |
| GC analysis, % | | | |
| Isoprene | 0 | 2 | 4 |
| Limonene/Carvestrene | 76 | 73 | 71 |
| 4-ethenyl-1,4-dimethyl | 16 | 15 | 13 |
| cyclohexene | | | |
| Cyclooctadienes | 5 | 7 | 8 |
| Terpinolenes | 3 | 3 | 2 |
| Others | 0 | 0 | 2 |

As can be seen, the heat transfer fluid can be repeatedly used without the loss of its activity, or compromising on the selectivity of the products.

### Example 13

Synthesis of Cyclic Alkanes from Terpenes Produced from Mevalonolactone

Monoterpenes produced from the second step of the reaction sequence (for instance, the product from Example 7) were further hydrogenated to produce various cyclic alkanes. The hydrogenation reaction of terpenes to cycloalkanes was performed in 300 mL Parr reactors under neat conditions using 150 mL of terpenes as a feed and using 1-2 g of a catalyst. The solution was then stirred at 1000 RPM and purged several times with N₂ and H₂ at room temperature. After this cycle of purging, the mixture was heated to 120° C. under a pressure of 800-1200 psi of H₂ while being stirred. The mixture was held at semi-batch reaction conditions for 8-12 hours with intermittent addition of H₂ gas to maintain the system at 1200 psi. The catalysts used for the hydrogenation reaction included Pd/C, Pd/NbOPO₄ and Ru/NbOPO₄. Samples were taken at different time intervals to check the progress of the reaction and after the reaction was completed, the reactor was allowed to cool to room temperature, depressurized, and the liquid samples were centrifuged and analyzed using GC-FID and GC-MS. The results are shown in table 10. In a similar way, the bicyclic C15 sesquiterpenes and C20 diterpenes, produced through the acid catalyzed condensation reaction of monoterpenes with isoprene as well as by the dimerization reaction of C10 monoterpenes, can be selectively converted to the respective bicyclic alkanes under similar hydrogenation reaction conditions.

TABLE 10
Analysis of liquid products by GC-FID and GC-MS obtained
in the hydrogenation of terpenes synthesized by Diels-
Alder reaction to various cyclic alkanes.

| Catalysts | Pd/C | Pd/NbOPO₄ | Ru/NbOPO_{₄} | Pd/Al₂O₃ |
|---|---|---|---|---|
| Batch time, h | 10 | 10 | 10 | 10 |
| Pressure, psi | 1200 | 1200 | 1200 | 1200 |
| T, ° C. | 120 | 120 | 120 | 120 |
| Main products | | | | |
| GC analysis, % | | | | |
| p-menthane | 42 | 38 | 37 | 35 |
| carvestrane | 36 | 35 | 33 | 33 |
| 4-ethyl-1,4-dimethyl | 17 | 19 | 18 | 16 |
| cyclohexane | | | | |
| dimethyl cyclooctanes | 5 | 7 | 10 | 10 |
| Others | 0 | 1 | 2 | 6 |

The reaction mixture of the invention and the method of the invention can thus be used to obtain hydrogenated cycloalkanes with the minimum number of steps involved in the conversion with high selectivity of the relevant intermediate compounds and consequently improving the final yields. Such a product rich in aromatics is labeled as 100% AroSAF and may be used as a blend component.

### Example 14

Synthesis of Aromatics and Cycloalkanes from Terpenes Produced from Mevalonolactone without Hydrogen Addition (AroSAF)

Monoterpenes produced from the second step of the reaction sequence (for instance, the product from Example 7) were further dehydrogenated to produce various substituted aromatics. Surprisingly, cycloalkanes were generated as a minor product, in-situ, i.e. hydrogenation of part of the monoterpenes progressed without addition of any external hydrogen. An aromatization reaction of terpenes was performed in 300 mL Parr reactors under neat conditions using 150 mL of terpenes as a feed and using 0.5-1 g of a catalyst without using hydrogen. The solution was then stirred at 1000 RPM and purged several times with N₂ at room temperature. After this cycle of purging, the mixture was heated to 250° C. while being stirred. The mixture was held at reaction conditions for 5 hours. The catalysts used for the reaction included various Pt, Pd, Co, Ru doped carbon, silica and alumina catalysts. Samples were taken at different time intervals to check the progress of the reaction and after the reaction was completed, the reactor was allowed to cool to room temperature, and the liquid samples were centrifuged and analyzed using GC-FID and GC-MS. The results are shown in table 11.

TABLE 11
Analysis of liquid products by GC-FID and GC-MS obtained
in the aromatization reaction of terpenes synthesized
by Diels-Alder reaction to various aromatics.

| Catalysts | Pd/C | Pt/C | Ru/C | Pd/Al₂O₃ |
|---|---|---|---|---|
| Batch time, h | 10 | 10 | 10 | 10 |
| T, ° C. | 250 | 250 | 250 | 250 |
| Main Products | | | | |
| GC analysis, % | | | | |
| p-cymene | 34 | 26 | 25 | 31 |
| m-cymene | 29 | 24 | 23 | 26 |
| 4-ethyl-1,4-dimethyl | 17 | 15 | 15 | 17 |
| cyclohexane | | | | |
| p-menthane | 13 | 20 | 22 | 18 |
| dimethyl cyclooctanes | 5 | 5 | 4 | 4 |
| Others | 2 | 10 | 11 | 4 |

The reaction mixture of the invention and the method of the invention can thus be used to obtain aromatics and cycloalkanes with the minimum number of steps involved in the conversion with high selectivity of the relevant intermediate compounds and consequently improving the final yields without the need for additional hydrogen. Such a product rich in aromatics is labeled as 100% AroSAF and may be used as a blend component.

### Example 15

Synthesis of Aromatic Blend from Terpenes Produced from Mevalonolactone

Monoterpenes produced from the second step of the reaction sequence (for instance, the product from Example 7) were further dehydrogenated to produce various substituted aromatics in hydrocarbon solvents (C8-C15) produced though ATJ, FT, or HEFA. The FT hydrocarbon mixture possess ˜70% of n-paraffins and ˜30% of iso-paraffins having C9-C11 as main components while the HEFA mixture comprises of ˜20% n-paraffins and ˜80% of iso-paraffins with C9-C16 compounds as the main components. Using HEFA and FT C8-C16 hydrocarbons as solvents, C7 hydrocarbons were avoided due to their higher vapour pressure being unattractive in a finished fuel composition. fuel compositions having n- and iso-paraffins, cyclic alkanes and aromatics with varied compositions can be produced in one-step. Aromatization reaction of terpenes to cycloalkanes was performed in 300 mL Parr reactors using 50 mL of FT or HEFA mixture and using 50 mL of terpenes as a feed and using 0.5 g of a catalyst. The solution was then stirred at 1000 RPM and purged several times with N₂ at room temperature. After this cycle of purging, the mixture was heated to 250° C. while being stirred. The mixture was held at semi-batch reaction conditions for 5 hours. The catalysts used for the reaction included various Pt, Pd, Co, Ru doped carbon, silica and alumina catalysts. Samples were taken at different time intervals to check the progress of the reaction and after the reaction was completed, the reactor was allowed to cool to room temperature, and the liquid samples were centrifuged and analyzed using GC-FID and GC-MS. Results showed the presence of ˜30% aromatics and ˜10% of cyclic alkanes together with the n- and iso-paraffins. Further by simply tuning the ratio of HEFA/FT solvents to the terpene mixture, various fuel surrogates with aromatics content from 1-30% can easily be synthesized.

### Example 16

Synthesis of Cyclic Alkanes from Terpenes Produced from Mevalonolactone

Monoterpenes produced from the second step of the reaction sequence were further hydrogenated to produce various cyclic alkanes. The hydrogenation reaction of terpenes to cycloalkanes was performed in 300 mL Parr reactors under neat conditions using 150 mL of terpenes as a feed and using 0.5-1 g of a catalyst. The solution was then stirred at 1000 RPM and purged several times with N₂ and H₂ at room temperature. After this cycle of purging, the mixture was heated to 120° C. under a pressure of 800-1200 psi of H₂ while being stirred. The mixture was held at semi-batch reaction conditions for 8-12 hours with intermittent addition of H₂ gas to maintain the system at 1200 psi. The catalysts used for the hydrogenation reaction included Pd/C, Pd/NbOPO₄ and Ru/NbOPO₄. Samples were taken at different time intervals to check the progress of the reaction and after the reaction was completed, the reactor was allowed to cool to room temperature, depressurized, and the liquid samples were centrifuged and analyzed using GC-FID and GC-MS. The results are shown in table 12.

TABLE 12
Analysis of liquid products by GC-FID and GC-MS obtained
in the hydrogenation of terpenes synthesized by Diels-
Alder reaction to various cyclic alkanes.

| Catalysts | Pd/C | Pd/NbOPO₄ | Ru/NbOPO₄ | Pd/Al₂O₃ |
|---|---|---|---|---|
| Batch time, h | 10 | 10 | 10 | 10 |
| Pressure, psi | 1200 | 1200 | 1200 | 1200 |
| T, ° C. | 120 | 120 | 120 | 120 |
| Main products | | | | |
| GC analysis, % | | | | |
| p-menthane | 42 | 38 | 37 | 35 |
| carvestrane | 36 | 35 | 33 | 33 |
| 4-ethyl-1,4-dimethyl | 17 | 19 | 18 | 16 |
| cyclohexane | | | | |
| dimethyl cyclooctanes | 5 | 7 | 10 | 10 |
| Others | 0 | 1 | 2 | 6 |

The reaction mixture of the invention and the method of the invention can thus be used to obtain hydrogenated cycloalkanes with the minimum number of steps involved in the conversion with high selectivity of the relevant intermediate compounds and consequently improving the final yields.

### Example 17

Synthesis of Cyclic Alkanes without any Aromatics from Terpenes Produced from Mevalonolactone

Monoterpenes produced from the second step of the reaction sequence were further hydrogenated to produce various cyclic alkanes without making any aromatics. The hydrogenation reaction of terpenes to cycloalkanes was performed in 300 mL Parr reactors under neat conditions using 150 mL of terpenes as a feed and using 1-2 g of a Ni based catalyst (NiSAT 300). The solution was then stirred at 1000 RPM and purged several times with N₂ and H₂ at room temperature. After this cycle of purging, the mixture was heated to 200-250° C. under a pressure of 800-1500 psi of H₂ while being stirred. The mixture was held at semi-batch reaction conditions for 8-12 hours with intermittent addition of H₂ gas to maintain the system at 1200 psi. Samples were taken at different time intervals to check the progress of the reaction and after the reaction was completed, the reactor was allowed to cool to room temperature, depressurized, and the liquid samples were centrifuged and analyzed using GC-FID and GC-MS. The GC MS results are shown in table 13 which showed nearly 28% of ethyl-dimethyl cyclohexanes, 67% of menthanes and 5% of dimethyl cyclooctanes.

TABLE 13
GC-MS Analysis of liquid products obtained in the hydrogenation of terpenes synthesized
by Diels-Alder reaction to exclusively cyclic alkanes at 200 C. and 1000 psi H2 pressure.

| | Cycloalkane Compound | | | | |
|---|---|---|---|---|---|
| | | | Ethyl-dimethyl | Dimethyl | |
| | p-Methane | Carvestrane | cyclohexane | Cyclooctanes | Others |
| | Percentage Composition | | | | |
| | 27 | 39.8 | 28 | 5 | 0.21 |
| RT | Area | Formula | m/z | Name | Area % |
| 5.001 | 198882 | C9 H18 | 97 | 1-Ethyl-3-methylcyclohexane (ct) | 0.087409162 |
| 5.22 | 64732 | C9 H18 | 97 | 1-Ethyl-3-methylcyclohexane (ct) | 0.028449884 |
| 5.254 | 126655 | C9 H18 | 97 | 1-Ethyl-3-methylcyclohexane (ct) | 0.055665206 |
| 5.568 | 93550 | C10 H18 | 95 | Bicyclo[2.2.1]heptane, 1,7,7-trimethyl- | 0.041115471 |
| 5.704 | 12808006 | C10 H20 | 111 | Cyclohexane, 1-ethyl-1,3-dimethyl-, trans- | 5.529152322 |
| 5.782 | 19697662 | C10 H20 | 111 | Cyclohexane, 1-ethyl-1,4-dimethyl-, cis- | 8.657174254 |
| 5.832 | 11228173 | C10 H20 | 111 | Cyclohexane, 1-ethyl-1,3-dimethyl-, trans- | 4.934811564 |
| 5.862 | 21328434 | C10 H20 | 111 | Cyclohexane, 1-ethyl- 1,4-dimethyl-, cis- | 9.373902837 |
| 5.942 | 58710727 | C10 H20 | 97 | Cyclohexane, 1-methyl-4-(3-methylethyl)-, cis- | 25.80351893 |
| 5.976 | 31513388 | C10 H20 | 97 | Cyclohexane, 1-methyl-4-(3-methylethyl)-, cis- | 13.85021691 |
| 6.076 | 26937130 | C10 H20 | 97 | Cyclohexane, 1-methyl-4-(3-methylethyl)-, trans- | 11.83893948 |
| 6.125 | 33302803 | C10 H20 | 97 | Cyclohexane, 1-methyl-4-(1-methylethyl)-, trans- | 14.6366695 |
| 6.194 | 36484 | C10 H20 | 97 | Cyclohexane, 1-methyl-4-(1-methylethyl), trans- | 0.016034814 |
| 6.238 | 102768 | C10 H20 | 97 | Cyclohexane, 1-methyl-4-(1-methylethyl)-, trans- | 0.045166806 |
| 6.528 | 2953135 | C10 H20 | 55 | Cyclooctane, 1,5-dimethyl- | 1.297910599 |
| 6.589 | 8427395 | C10 H20 | 55 | Cyclooctane, 1,5-dimethyl- | 3.703862266 |

The reaction mixture of the invention and the method of the invention can thus be used to obtain hydrogenated cycloalkanes with the minimum number of steps involved in the conversion with high selectivity of the relevant intermediate compounds and consequently improving the final yields. Such a product rich in cycloalkanes with very low to non-detectable aromatics is labeled as 100% CA-SAF and may be used as a blend component or neat fuel.

### Example 18

Synthesis of Aromatics from Terpenes Produced from Mevalonolactone

Monoterpenes produced from the second step of the reaction sequence were further dehydrogenated to produce various substituted aromatics. An aromatization reaction of terpenes to cycloalkanes was performed in 300 mL Parr reactors under neat conditions using 150 mL of terpenes as a feed and using 0.5-1 g of a catalyst without using hydrogen. The solution was then stirred at 1000 RPM and purged several times with N₂ at room temperature. After this cycle of purging, the mixture was heated to 250° C. while being stirred. The mixture was held at semi-batch reaction conditions for 5 hours. The catalysts used for the reaction included various Pt, Pd, Co, Ru doped carbon, silica and alumina catalysts. Samples were taken at different time intervals to check the progress of the reaction and after the reaction was completed, the reactor was allowed to cool to room temperature, and the liquid samples were centrifuged and analyzed using GC-FID and GC-MS. The results are shown in table 14.

TABLE 14
Analysis of liquid products by GC-FID and GC-MS obtained
in the aromatization reaction of terpenes synthesized
by Diels-Alder reaction to various aromatics.

| Catalysts | Pd/C | Pt/C | Ru/C | Pd/Al₂O₃ |
|---|---|---|---|---|
| Batch time, h | 10 | 10 | 10 | 10 |
| T, ° C. | 250 | 250 | 250 | 250 |
| Main Products | | | | |
| GC analysis, % | | | | |
| p-cymene | 34 | 26 | 25 | 31 |
| m-cymene | 29 | 24 | 23 | 26 |
| 4-ethyl-1,4-dimethyl | 17 | 15 | 15 | 17 |
| cyclohexane | | | | |
| p-menthane | 13 | 20 | 22 | 18 |
| dimethyl cyclooctanes | 5 | 5 | 4 | 4 |
| Others | 2 | 10 | 11 | 4 |

The reaction mixture of the invention and the method of the invention can thus be used to obtain aromatics with the minimum number of steps involved in the conversion with high selectivity of the relevant intermediate compounds and consequently improving the final yields. These aromatics can be used suitably as blends and/or additives for fuels.

### Example 19

Synthesis of Aromatic Blend from Terpenes Produced from Mevalonolactone

Monoterpenes produced from the second step of the reaction sequence were further dehydrogenated to produce various substituted aromatics in hydrocarbon solvents (C8-C15) produced though Fischer Tropsch (FT) or Alcohol to Jet (ATJ) or Hydrotreating of Esters and Fatty acids (HEFA) or combination thereof. The FT hydrocarbon mixture possess ˜70% of n-paraffins and ˜30% of iso-paraffins having C9-C11 as main components while the HEFA mixture comprises of ˜20% n-paraffins and ˜80% of iso-paraffins with C9-C16 compounds as the main components. Using HEFA and FT C8-C15 hydrocarbons as solvents, C7 hydrocarbons were avoided due to their higher vapour pressure being unattractive in a finished fuel composition. fuel compositions having n- and iso-paraffins, cyclic alkanes and aromatics with varied compositions can be produced in one-step. Aromatization/Hydrogenation reaction of terpenes to cycloalkanes was performed in 300 mL Parr reactors using 50 mL of FT or HEFA mixture and using 50 mL of terpenes as a feed and using 0.5 g of a catalyst. The solution was then stirred at 1000 RPM and purged several times with N₂ at room temperature. After this cycle of purging, the mixture was heated to 250° C. while being stirred. The mixture was held at semi-batch reaction conditions for 5 hours. The catalysts used for the reaction included various Pt, Pd, Co, Ru doped carbon, silica and alumina catalysts. Samples were taken at different time intervals to check the progress of the reaction and after the reaction was completed, the reactor was allowed to cool to room temperature, and the liquid samples were centrifuged and analyzed using GC-FID and GC-MS. Results showed the presence of ˜30% aromatics and ˜10% of cyclic alkanes together with the n- and iso-paraffins. Further by simply tuning the ratio of HEFA/FT solvents to the terpene mixture, various fuel surrogates with aromatics content from 5-30% can easily be developed.

### Example 20

A sustainable fuel composition was prepared according to Example 14-15, using HEFA as the solvent. The reaction mixture was then further diluted using the same HEFA material to give compositions with 21% w/w aromatic compounds, 15% w/w aromatic compounds and 10% w/w aromatic compounds. These fuel compositions were analysed by gas-liquid chromatography with the results as shown in Table 15. The results confirm the substantial absence of PAHs in the fuel compositions.

TABLE 15
Analysis of fuel compositions produced from mevalanolactone in
which HEFA is used as a solvent and diluent for the final composition,
indicating the substantial absence of PAH compounds.

| Class of compound | % by GC | % by GC | % by GC |
|---|---|---|---|
| n and iso paraffins | 79.92 | 72.36 | 63.26 |
| cycloaliphatic hydrocarbons | 8.67 | 12.09 | 15.45 |
| mono aromatic hydrocarbons | 10.39 | 15.55 | 21.27 |
| poly aromatic hydrocarbons | 0.06 | 0.00 | 0.00 |
| Total | 99.03 | 100.00 | 99.99 |

A more detailed analysis of the composition with the highest aromatic content is shown in Table 16, showing that the PAH content was below the detection limit.

TABLE 16
Detailed analysis of the fuel composition.

| Compound | % w/w by GC | Compound | % w/w by GC |
|---|---|---|---|
| Alkylbenzenes C08 | 0.07 | Biphenyls C16 | 0.00 |
| Alkylbenzenes C09 | 0.12 | Naphthalenes C16 | 0.00 |
| Alkylbenzenes C10 | 21.08 | Dicycloparaffins C10 | 7.21 |
| Alkylbenzenes C11 | 0.01 | Dicycloparaffins C11 | 0.02 |
| Cycloaromatics C17 | 0.00 | Dicycloparaffins C18 | 0.00 |
| Cycloaromatics C18 | 0.00 | Dicycloparaffins C19 | 0.00 |
| Monocycloparaffins C08 | 0.46 | iso-Paraffins C05 | 0.01 |
| Monocycloparaffins C09 | 0.78 | iso-Paraffins C06 | 0.02 |
| Monocycloparaffins C10 | 6.90 | iso-Paraffins C07 | 0.19 |
| Monocycloparaffins C11 | 0.08 | iso-Paraffins C08 | 1.95 |
| Monocycloparaffins C12 | 0.00 | iso-Paraffins C09 | 7.16 |
| Monocycloparaffins C20 | 0.00 | iso-Paraffins C10 | 7.99 |
| n-paraffins C6 | 0.03 | iso-Paraffins C11 | 7.46 |
| n-paraffins C7 | 0.17 | iso-Paraffins C12 | 5.05 |
| n-paraffins C8 | 1.97 | iso-Paraffins C13 | 3.89 |
| n-paraffins C9 | 2.61 | iso-Paraffins C14 | 5.58 |
| n-paraffins C10 | 2.11 | iso-Paraffins C15 | 4.26 |
| n-paraffins C11 | 1.14 | iso-Paraffins C16 | 8.00 |
| n-paraffins C12 | 0.55 | iso-Paraffins C17 | 1.28 |
| n-paraffins C13 | 0.28 | iso-Paraffins C18 | 1.05 |
| n-paraffins C14 | 0.30 | | |
| n-paraffins C15 | 0.08 | | |
| n-paraffins C16 | 0.12 | | |
| Total | 100.00 | | |

Thus, it can be seen that the reactant mixture of the invention and the method of the invention enables production of suitable alkanes, isoalkanes, cycloalkanes with the minimum number of steps with high level of selectivity and increased yields, wherein the final products includes the monoaromatic hydrocarbons at a desirable levels while the polyaromatic hydrocarbons are below detectable levels.

### Example 21

Separation of Cycloalkanes from an Aromatics-Cycloalkane Fuel Mixture Using an Extractive Distillation Procedure

In another approach, the cycloalkane-aromatic fuel blend produced as shown in Example 9, were further treated for an extractive distillation procedure to separate cycloalkanes from the aromatics feed and to increase the aromatic content in the fuel mixture. We have used various high boiling ester-, alcohol- and ether-containing organic solvents such as butyl benzoate, undecyl alcohol, tripropylene glycol methyl ether, butyldiglycolether, etc. to perform the extractive distillation and to increase the cycloalkane content in various distilled fractions. For instance, we used an equivolume mixture of aromatics-cycloalkane fuel blend and butyl benzoate (100 ml each) and collected different fractions at different temperatures at <10 mbar pressures (Table 17). By this procedure, we were able to produce higher concentration aromatics (over 99%, F7) and double the concentration of cycloalkanes (F1 & F2) from the original cycloalkane-aromatic fuel blend (60% aromatics and 40% cycloalkanes) as shown in Example 9. We also conducted a second round of distillations by combining fractions (F1 and F2) which showed that the cycloalkane content can be further increased to >85%.

Table 17. Extractive Distillation Using Butyl Benzoate as Solvent to Separate Cycloalkanes from the Aromatic-Cycloalkane Fuel Blend.

(a) First Round of Distillation
Using Butyl Benzoate (BuBz) as Solvent, Feed Used:
96 g + 100 g BuBz, P = < 10 mbar

| | |
|---|---|
| Fraction 1 (F1, 60C, ~4 g) | 30% Aromatics + 70% Cycloalkanes |
| Fraction 2 (F2, 65C, ~12 g) | 35% Aromatics + 65% Cycloalkanes |
| Fraction 3 (F3, 67C, ~28 g) | 49% Aromatics + 51% Cycloalkanes |
| Fraction 4 (F4, 69C, 15 g) | 50% Aromatics + 50% Cycloalkanes |
| Fraction 5 (F5, 70C, 8.5 g) | 61% Aromatics + 39% Cycloalkanes |
| Fraction 6 (F6, 75-80C, 10 g) | 85% Aromatics + 15% Cycloalkanes |
| Fraction 7 (F7, in reaction pot, 19 g) | 99.5% Aromatics + 0.5% Cycloalkanes |

(b) Second Round of Distillation
Using Butyl Benzoate (BuBz) as Solvent, Feed Used:
15 g (F1 + F2) + 300 g BuBz, P = < 10 mbar

| | |
|---|---|
| Fraction 1 (F1, 59C, ~1 g) | 14% Aromatics + 86% Cycloalkanes |
| Fraction 2 (F2, 60C, ~3.5g) | 15% Aromatics + 85% Cycloalkanes |
| Fraction 3 (F3, 65-70C, ~5g) | 28% Aromatics + 72% Cycloalkanes |

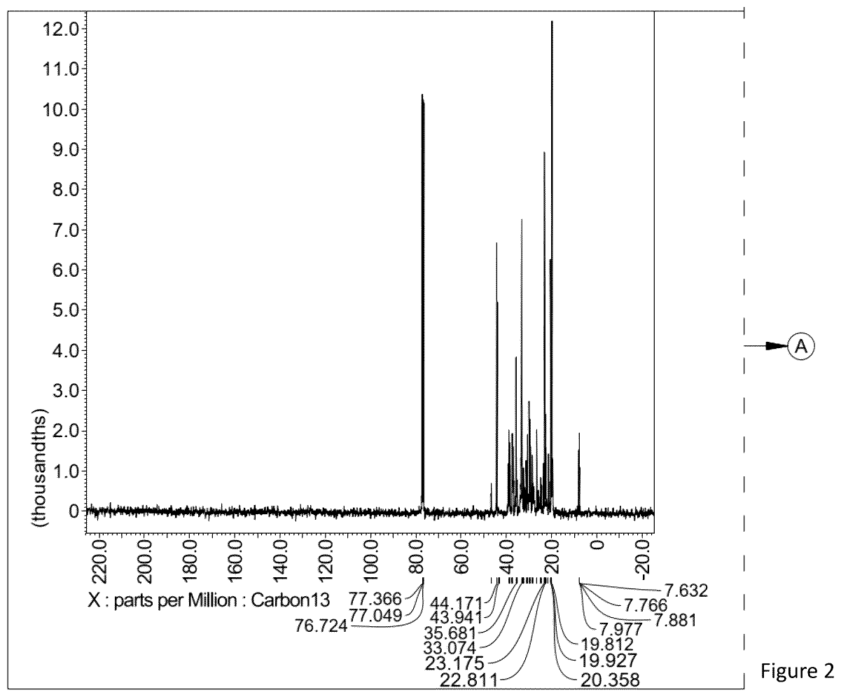

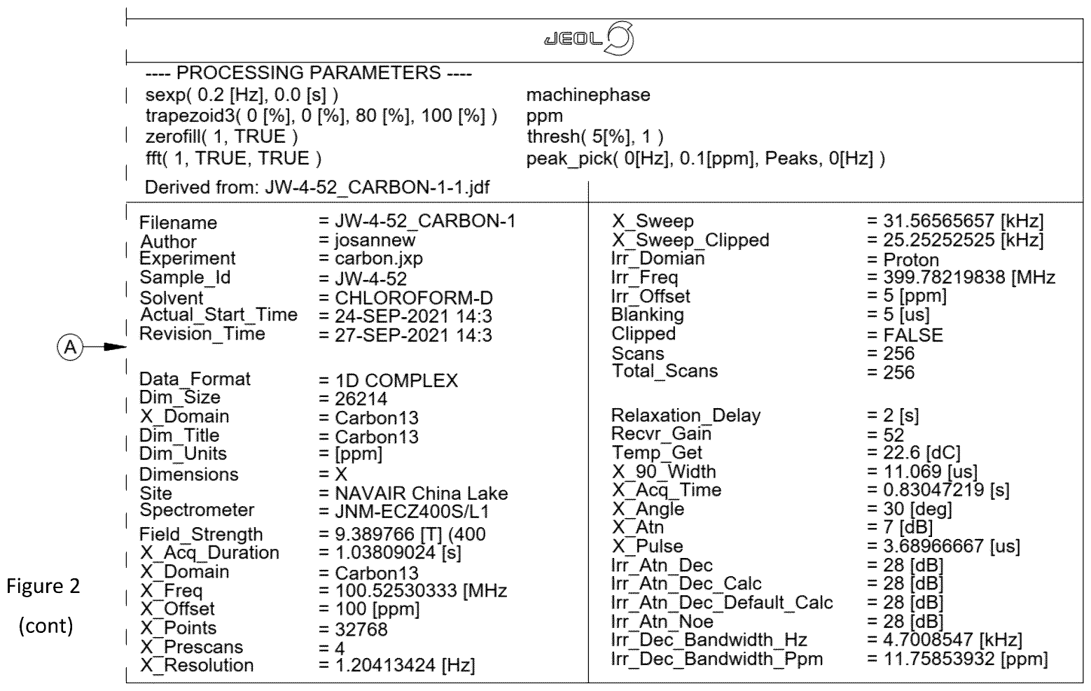

Fractionation can be continued to give very high levels of cycloalkanes, approaching 100% as evidenced by the proton and carbon 13 NMR spectra shown in FIGS. 1 and 2 respectively.

### Example 22

### Improving Dimethyl Cyclooctane (DMCO) Content in the Fuel Mixture

Dimethyl cyclooctanes are known to have higher energy densities than many other hydrocarbons, hence improving the percentage of DMCO is beneficial from a fuel energy density perspective. As shown in Example 12, we have utilized an extractive distillation procedure to increase the cycloalkane content in the cycloalkane-aromatic fuel mixture. By using this procedure, the DMCO content of ˜5-7% in the original fuel mixture was increased to ˜15-20% in the distilled sample.

### Example 23

### Reaction of Isoprene and Olefins to Give a Mixture of Monoterpenes, Alkylcyclohexenes and Other Hydrocarbons in Presence or Absence of Catalysts

Reactions were performed in 300 mL Parr reactors using isoprene and a suitable olefin or mixture of olefins as a feed and a heat transfer liquid as solvent in the presence and absence of various acid catalysts. Tests were performed with 100 g of heat transfer liquid and an equal volume of a mixture of isoprene and 1-hexene in the presence of selected catalysts. Catalysts used included silicon dioxide, aluminum oxide, silica-alumina and titania. The ratio of isoprene to olefin was adjusted to balance the relative reaction rates of the reaction isoprene plus isoprene and the reaction isoprene plus 1-hexene. (For different olefins this would depend for example on the electron donating nature of the alkyl group(s) present on the olefin(s).) The reaction mixture was heated to a temperature in the range from 150 to 250° C. while being stirred at 1000 RPM. The temperature chosen affected the balance of products and so temperature can be chosen to optimise the desired product mixture. After completion of the reaction, the reactor was allowed to cool to room temperature, depressurized, and the liquid samples were centrifuged and the liquid sample was analyzed using gas chromatography (GC-FID) and high-performance liquid chromatography (HPLC). These alkylcyclohexenes can be processed to make aromatics and cycloalkanes in a similar way to highlighted in prior examples like Examples 13, 14 and 15.

TABLE 18
Analysis of liquid products by GC-FID and GC-MS obtained
from the conversion of isoprene and 1-hexene (in equimolar
ratio) in the absence/presence of different catalysts.

| | Reaction time, h | | | | |
|---|---|---|---|---|---|
| | 5 | 5 | 5 | 5 | 5 |
| | Catalyst | | | | |
| GC analysis, % | None | Silica | Alumina | Titania | Si—Al |
| Isoprene | minor | minor | minor | minor | minor |
| Limonene/Carvestrene | yes | yes | yes | yes | yes |
| 4-ethenyl-1,4- | yes | yes | yes | yes | yes |
| dimethyl cyclohexene | | | | | |
| Cyclooctadienes | minor | minor | minor | minor | minor |
| Terpinolenes | minor | minor | minor | minor | yes |
| 1-alkene(s) | 0 or | 0 or minor | 0 or minor | 0 or minor | 0 or minor |
| | minor | | | | |
| 1-methyl-4- | yes | yes | yes | yes | yes |
| butylcyclohex-1- | | | | | |
| ene(s) | | | | | |
| 2-methyl-4- | yes | yes | yes | yes | yes |
| butylcyclohex-1- | | | | | |
| ene(s) | | | | | |
| Others | minor | minor | minor | minor | minor |

### Example 24 (Prophetic)

### Reaction of Butadiene and Olefins to Give a Mixture of Alkylcyclohexenes and Other Hydrocarbons in Presence or Absence of Catalysts

Reactions are performed in 300 mL Parr reactors using butadiene and a suitable olefin or mixture of olefins as a feed and a heat transfer liquid as solvent in the presence and absence of various acid catalysts. Tests are performed with 100 g of heat transfer liquid and an equal volume of a mixture of butadiene and olefin(s) in the presence of selected catalysts such as silicon dioxide, aluminum oxide, silica-alumina, titania and niobia. The ratio of butadiene to olefin is adjusted to balance the relative reaction rates of butadiene plus butadiene and butadiene plus olefin. (This would depend for example on the electron donating nature of the alkyl group(s) present on the olefin(s).) The reaction mixture is heated to a suitable temperature, for example in the range from 150 to 250° C. while being stirred at 1000 RPM. The temperature choice would affect the balance of products and so temperature would be chosen to optimize the desired product mixture. After completion of the reaction, the reactor is allowed to cool to room temperature, depressurized, and the liquid samples are centrifuged and the liquid sample are analyzed using gas chromatography (GC-FID) and high-performance liquid chromatography (HPLC).

Sources of olefins for performing this example include:
a) Individual olefins derived from ethylene oligomerization such as 1-hexene, 1-octene.
b) Mixed olefins derived from ethylene oligomerization such as a mix of butene, hexene, octene and decene.
c) Mixed olefins derived or accessed from the Fischer Tropsch (FT) style oligomerization process directly or indirectly from longer chain FT hydrocarbons via a combination of cracking, distillation and dehydrogenation such as a mix of C3 to C10 olefins.
d) Mixed olefins derived from a combination of cracking, distillation and dehydrogenation of hydrocarbons obtained from hydroprocessed esters and fatty acids such as a mix of C3 to C10 olefins.
e) Olefins derived from dehydrogenation of natural gas liquids such as C3 to C6 olefins.

TABLE 19
Anticipated Analysis of liquid products by GC-FID and GC-MS
obtained from the conversion of Butadiene and mixed alpha
olefins in the absence/presence of different catalysts.

| | Reaction time, h | | | | |
|---|---|---|---|---|---|
| | 5 | 5 | 5 | 5 | 5 |
| | Catalyst | | | | |
| GC analysis | None | Silica | Alumina | Titania | Si—Al |
| Butadiene | minor | minor | minor | minor | minor |
| 4-vinylcyclohexene | yes | yes | yes | yes | yes |
| Cyclooctadienes | minor | minor | minor | minor | minor |
| 4- | minor | minor | minor | minor | minor |
| ethylidenecyclohexene | | | | | |
| 3- | minor | minor | minor | minor | minor |
| ethylidenecyclohexene | | | | | |
| 1-alkenes | 0 or | 0 or minor | 0 or minor | 0 or minor | 0 or minor |
| | minor | | | | |
| 4-alkylcyclohex-1- | yes | yes | yes | yes | yes |
| enes | | | | | |
| Others | minor | minor | minor | minor | minor |

Note: the specific 1-alkenes and 4-alkylcyclohex-1-enes will depend on the particular choice of olefin or olefins as described for example in cases a, b, c, d and e above. These alkylcyclohexenes can be processed to make aromatics and cycloalkanes in a similar way to highlighted in prior examples like Examples 13, 14 and 15.

### Example 25

### Reaction of a Mixture of Alkylated Cyclohexenes and Other Hydrocarbons to Produce Aromatics

Alkylated cyclohexenes and other hydrocarbons produced from Example 14 and 15 reaction sequences were further dehydrogenated to produce various substituted aromatics. An aromatization reaction of Alkylated cyclohexenes and other hydrocarbons to aromatics and cycloalkanes was performed in 300 mL Parr reactors under neat conditions using 150 mL of Alkylated cyclohexenes and other hydrocarbons produced from Example 14 and 15 as a feed and using 0.5-1 g of a catalyst without using hydrogen. The solution was then stirred at 1000 RPM and purged several times with N₂ at room temperature. After this cycle of purging, the mixture was heated to 250° C. while being stirred. The mixture was held at semi-batch reaction conditions for 5 hours. The catalysts used for the reaction included various Pt, Pd, Co, Ru doped carbon, silica and alumina catalysts. Samples were taken at different time intervals to check the progress of the reaction and after the reaction was completed, the reactor was allowed to cool to room temperature, and the liquid samples were centrifuged and analyzed using GC-FID and GC-MS. The results are shown in table 20.

TABLE 20
Analysis of liquid products by GC-FID and GC-MS obtained in
the aromatization reaction of Alkylated cyclohexenes and
other hydrocarbons produced from Example 14 and 15 reaction
synthesized by Diels-Alder reaction to various aromatics.

| Catalysts | Pd/C | Pt/C | Ru/C | Pd/Al₂O₃ |
|---|---|---|---|---|
| Batch time, h | 5 | 5 | 5 | 5 |
| T, ° C. | 250 | 250 | 250 | 250 |
| Main Products | | | | |
| p-cymene (C10) | yes | yes | yes | yes |
| m-cymene | yes | yes | yes | yes |
| 4-ethyl-1,4-dimethyl | yes | yes | yes | yes |
| cyclohexane | | | | |
| p-menthane | yes | yes | yes | yes |
| dimethyl cyclooctanes | minor | minor | minor | minor |
| Alkylated benzenes | yes | yes | yes | yes |
| (C8-15) | | | | |
| Alkylated | yes | yes | yes | yes |
| cyclohexanes (C8-15) | | | | |
| Others | yes | yes | yes | yes |

### Example 26

Fuel Blends with 8-25% Aromatic Content, Minimum Density of 0.775 g/ml, Freeze Point Below −40° C. And Non-Detectable Polyaromatic Hydrocarbons

Products produced from Examples 14, 15 and 25 reaction sequences that are rich in alkylated benzenes (30-65%) along with co-produced alkylated cyclohexanes were further blended with hydrocarbons from other sources to produce final finished fuels with 8-25% aromatic content. Such final finished fuels also exhibit superior other properties like minimum density of 0.775 g/ml (at 15° C.) and freeze point below −40° C. Exemplary sources of hydrocarbons from other sources include those obtained from a FT process, HEFA process, ATJ process and jet fuel from petroleum. For instance, 72 ml of HEFA product (zero aromatics; 0.75 g/l density) was blended with 28 ml of product from example 14 (with composition listed in table 11) to obtain a final finished fuel with properties listed in table 21 (column 2, named AroSAF/HEFA 28/72). This final finished fuel is 100% renewable, as can be ascertained by carbon 12/carbon 14 ratio. Other renewable fuels compositions with high-performance can be obtained by mixing products produced from examples 14 (AroSAF) and example 17 (CA-SAF) in various ratios with other fuels like HEFA or petroleum derived Jet A. A multitude of these compositions along with improved properties are presented in table 21.

TABLE 21
Analysis of final fuel properties obtained by blending.

| | Inno- | | | | CA- | CA- | AroSAF/ | | |
|---|---|---|---|---|---|---|---|---|---|
| Name and | vative | AroSAF/ | AroSAF/ | AroSAF/ | SAF/ | SAF/ | HEFA/ | | |
| Composition | Fuel | HEFA | JetA | JetA | JetA | JetA | JetA | Pure | Pure |
| in vol % | Range | 28/72 | 10/90 | 20/80 | 10/90 | 20/80 | 10/40/50 | JetA | HEFA |
| Aromatic | 8-25% | 20% | — | — | — | — | — | — | 0 |
| content | | | | | | | | | |
| % p-cymene | 0.5-15% | 12% | — | — | — | — | — | — | 0 |
| % ethyl | 0.5-15% | 0% | — | — | — | — | — | — | 0 |
| benzene | | | | | | | | | |
| Density | | 0.775 | 0.786 | 0.792 | 0.783 | 0.785 | 0.774 | 0.780 | 0.751 |
| 15 C., g/cm3 | | | | | | | | | |
| Density 0 C., | | 0.785 | 0.798 | 0.804 | 0.794 | 0.796 | 0.786 | 0.791 | 0.762 |
| g/cm3 | | | | | | | | | |
| Density - | | 0.800 | 0.812 | 0.818 | 0.809 | 0.811 | 0.801 | 0.806 | 0.777 |
| 20 C., g/cm3 | | | | | | | | | |
| Density - | | 0.815 | 0.827 | 0.833 | 0.824 | 0.825 | 0.815 | 0.821 | 0.792 |
| 40 C., g/cm3 | | | | | | | | | |
| Viscosity | | 1.390 | 1.571 | 1.507 | 1.629 | 1.615 | 1.589 | 1.630 | 1.674 |
| 15 C., cSt | | | | | | | | | |
| Viscosity | | 1.767 | 2.045 | 1.949 | 2.119 | 2.117 | 2.062 | 2.146 | 2.205 |
| 0 C., cSt | | | | | | | | | |
| Viscosity - | | 2.761 | 3.302 | 3.131 | 3.427 | 3.402 | 3.315 | 3.476 | 3.557 |
| 20 C., cSt | | | | | | | | | |
| Viscosity - | | 4.921 | 6.178 | 5.781 | 6.452 | 6.346 | 6.228 | 6.613 | 6.840 |
| 40 C., cSt | | | | | | | | | |
| Surface | | 23.51 | 24.50 | 24.00 | 24.20 | 24.30 | 24.00 | 23.80 | 25.60 |
| Tension, | | | | | | | | | |
| mN/m | | | | | | | | | |
| Flash | | 39.0 | 42.5 | 43.5 | 42.5 | 42.5 | 41.5 | 42.0 | 39.0 |
| Point, C. | | | | | | | | | |
| Freeze | | −82.0 | −51.1 | −53.1 | −51.4 | −53.4 | −49.8 | −51.0 | −42.9 |
| Point, C. | | | | | | | | | |
| HHV, MJ/kg | | 46.59 | 46.45 | 46.17 | 46.61 | 46.46 | 46.69 | — | 47.30 |
| H %, mass | | 14.42 | 14.09 | 13.83 | 14.35 | 14.41 | 14.56 | — | 15.49 |
| LHV, MJ/kg | | 43.53 | 43.46 | 43.23 | 43.56 | 43.40 | 43.60 | 43.24 | 44.03 |

### Example 27

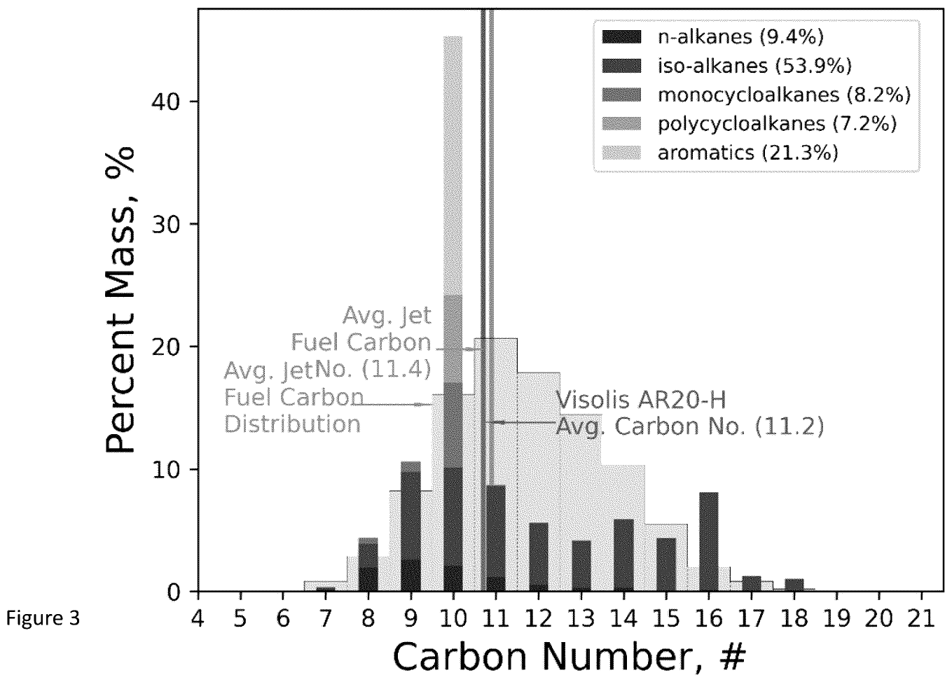

A fuel composition was synthesized according to the process of example 15. HEFA product was used as a solvent/diluent at 85% level. The 12 carbon aliphatic content of the final composition was around 5%. (see Figure. 3)

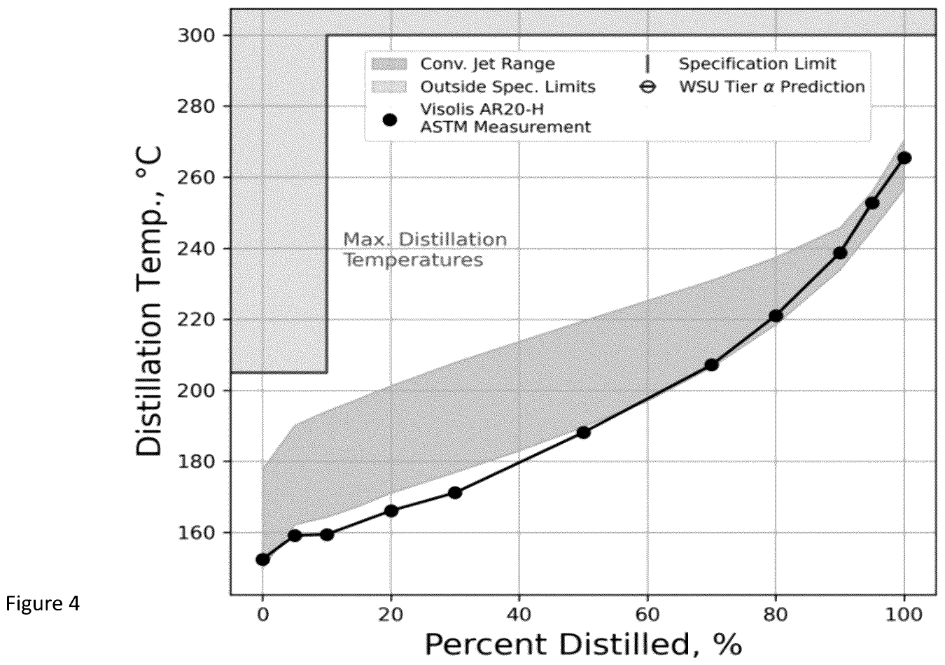

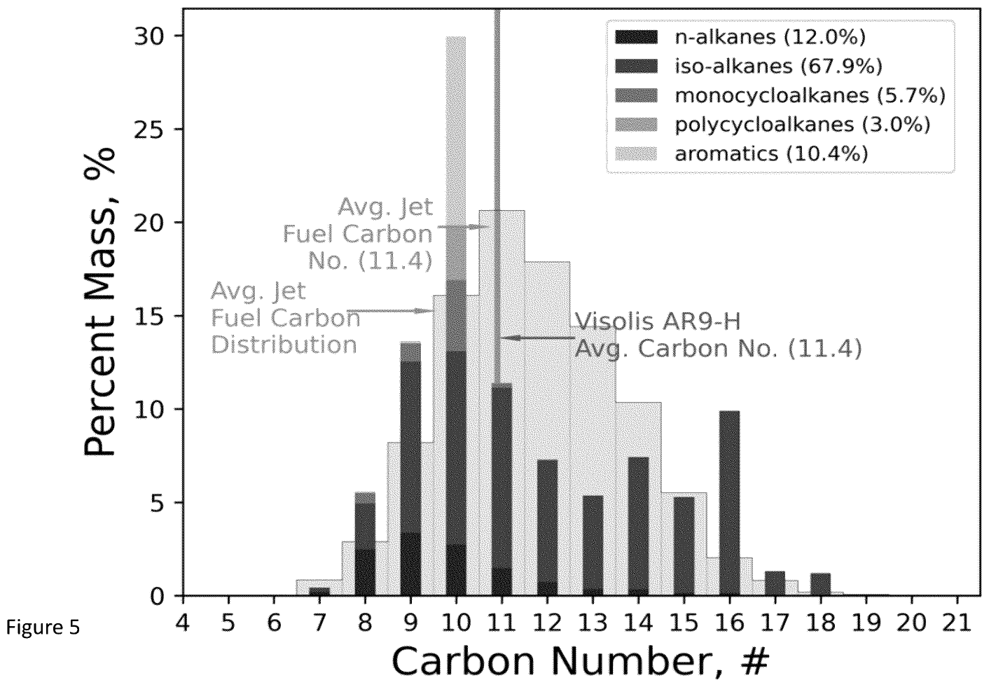

The composition had a particularly good distillation curve (see FIG. 4). At a different solvent ratio of 70% the composition obtained is shown in FIG. 5.

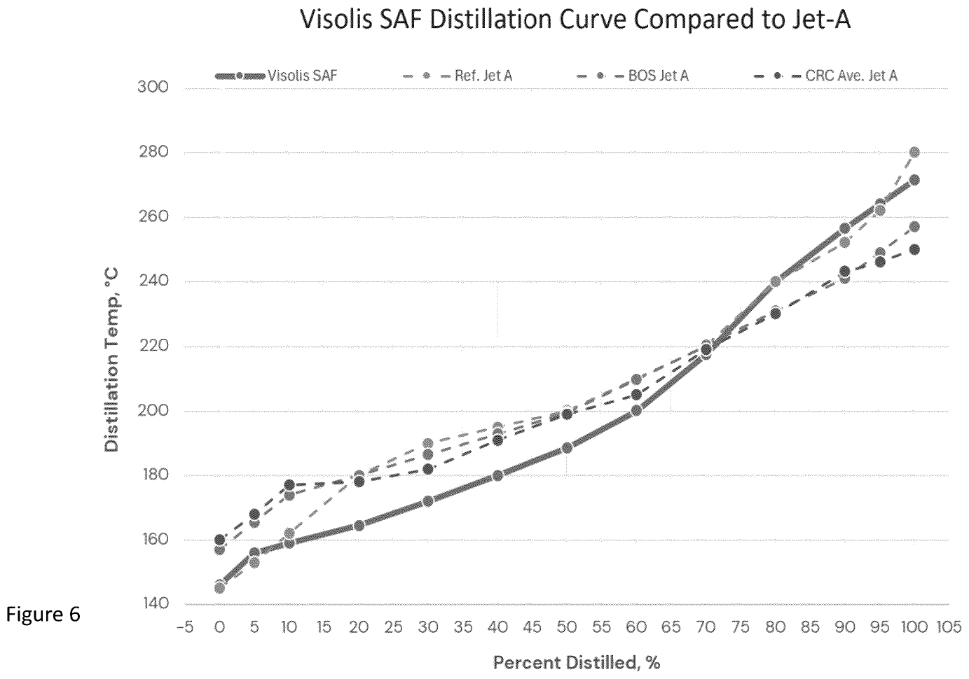

The distillation curve of the composition shown in FIG. 5 was measured and compared to the distillation curve of a sample of Jet A aviation fuel. The comparison is shown in FIG. 6.

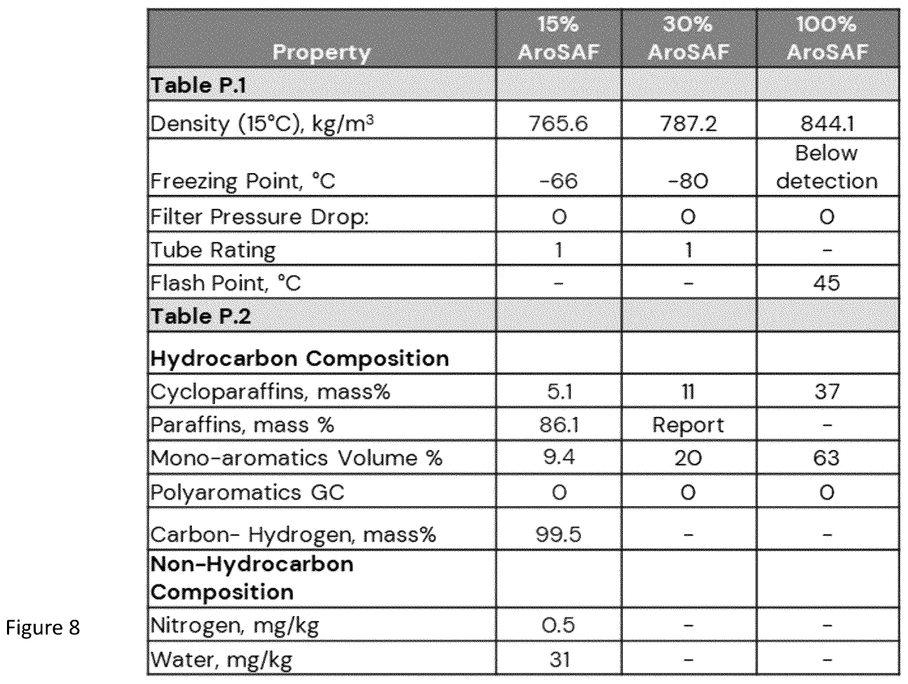

Various physical properties were measured for the samples of FIGS. 3 and 5. These results are shown in FIG. 8.

It can be seen from the data, that the composition in FIG. 5 has properties consistent with commercial Jet-A and use as a sustainable aviation fuel.

### Example 28

A fuel composition was prepared according to the method of example 14, with high aromatic content. The density of the composition at 15 C was 0.844 g/cm3. The lower heating value of the composition was 127495 BTU/US gallon. This is particularly high in comparison to the average heat content of aviation fuels given for example as 124070 BTU/US gallon in CRC World Fuel Survey, CRC Report No. 647, June 2006.

The sulfur content of this composition was measured and found to be less than 10 ppm.

### Example 29

A fuel composition was prepared according to the method of example 7, with high cycloaliphatic content. The density of the composition was 0.806 g/cm3 at 15 C. The lower heating value of the composition was 125388 BTU/US gallon.

The sulfur content of this composition was measured and found to be less than 10 ppm.

This composition has a volumetric heat content and density consistent with use as a rocket propellant such as propellant RP-1.

### Example 30

Comparative seal swelling tests were conducted using nitrile elastomer O rings using the method of Anuar et al. (See: Effect of fuels, aromatics and preparation methods on seal swell, Published online by Cambridge University Press: 12 Apr. 1 2021 A. Anuar, V. K. Undavalli, B. Khandelwal and S. Blakey).

A fuel composition with 65% monoaromatic content, comprising predominantly para and meta cymene was produced according to the method of example 14. This composition was further blended with various proportions of a mixture of hydrotreated esters and fatty acids (HEFA), typically used as a sustainable component when mixed with jet fuel. The resulting blends had an aromatic content of 5.2% w/w, 6.5% w/w, 9.75% w/w, 13.0% w/w and 19.5% w/w.

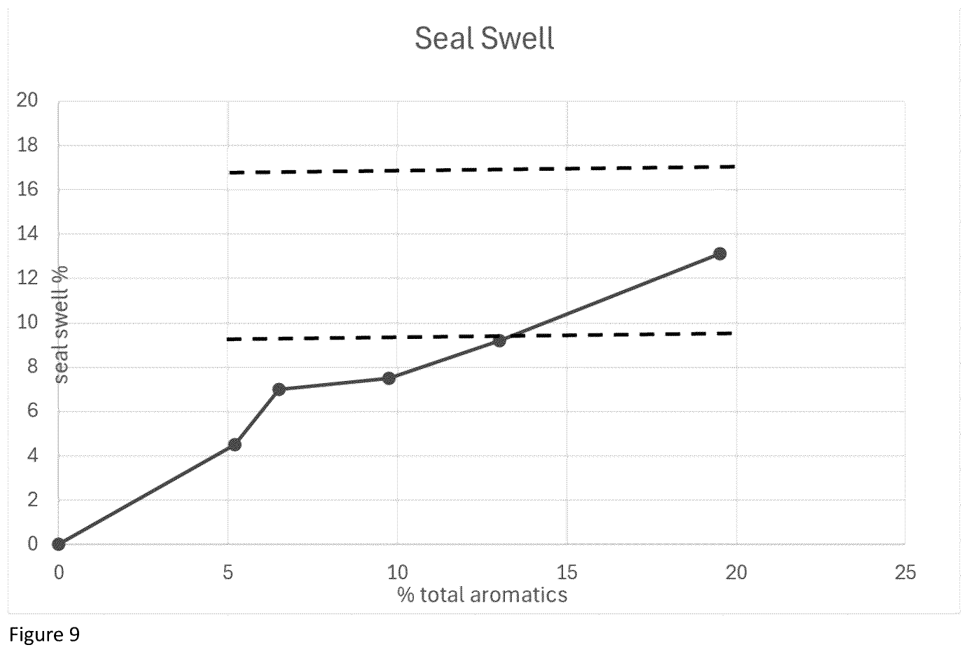

The HEFA component contributed essentially zero swelling capability. The swelling performance of the compositions were 4.5%, 7.0%, 7.5%, 9.2% and 13.1% respectively. demonstrating excellent swelling performance despite the absence of polyaromatic compounds. The results are shown graphically in FIG. 9 wherein the range of results obtained using the same test method with various samples of fossil Jet A1 are also clearly indicated. The data points show the relationship between % seal swell achieved and the % of total aromatics in the fully renewable blend. The dotted lines show the zone into which fossil Jet A1 samples fall.

### Example 31

A reaction mixture was prepared according to the method of Example 7. This mixture had the initial composition shown in column 1 of Table 22

TABLE 22
composition of starting material and distilled fractions.

| | | 2 | | | |
|---|---|---|---|---|---|
| | | Initial | 3 | 4 | 5 |
| GC analysis, % | 1 | distillation | Fraction 1 | Fraction 2 | Fraction 3 |
| Isoprene | 0 | 0 | 0 | 0 | 0 |
| Limonene/ | 65 | 67 | 41 | 97 | 65 |
| Carvestrene | | | | | |
| 4-ethenyl-1,4- | 15 | 24 | 57 | 1 | 0 |
| dimethyl | | | | | |
| cyclohexene | | | | | |
| Cyclooctadienes | 3 | 7 | 1 | 2 | 33 |
| Terpinolenes | 10 | | | | |
| Others | 7 | | | | |

Column 4 shows that fraction 2 was 97% w/w composed of Limonene and Carvestrene with minor amounts of other isoprene dimers like dimethylcyclooctadienes and terpinolenes.

Fraction 2 was mixed with 50% w/w of natural limonene. A control experiment using 100% natural limonene was also run. The test sample and the control sample were each mixed with an equivalent amount of styrene (terpene to styrene mass ratio 3 to 1) and the mixtures were polymerised with the use of a cationic catalyst.

The comparative results of the control and the test material are shown in Table 23.

TABLE 23

| | Control mixture | Fraction 2 test mixture |
|---|---|---|
| Yield | 89% | 94.5% |
| Isoprene dimers | 4.9% | 0.7% |
| Number average molar | 740 | 845 |
| mass (Mn) | | |
| Mass average molar | 1180 | 1845 |
| mass (Mw) | | |
| Softening point/C. | 112.5 | 121.5 |
| Glass transition | 66 | 74.5 |
| temperature Tg/C. | | |
| Colour value (APHA) | 150 | 150 |

It can be seen that the carvestrene/limonene fraction gives excellent yield and satisfactory polymerisation and other properties for use as a tackifier. Polymerisation conditions can be adapted to give lower molecular weights if desired. The combination of carvestrene and limonene outperforms limonene alone in polymerisation and yield.

The equivalent comparison using fraction 1 (dimethyl, ethylidene substituted cyclohexenes but with a substantial amount of limonene and carvestrene still present, gave somewhat lower molecular weights, see table 24. This indicates that apart from consideration of resin yield, it would seem unnecessary to separate fractions 1 and 2 for this application. The materials of fraction 1 have an enhanced tendency for chain termination and hence by controlling their amount, polymer performance can be tuned as desired. Furthermore, these sustainable polymer compositions comprised of Limonene and Carvestrene with certain amounts of other isoprene dimers like dimethylcyclooctadienes and terpinolenes can be produced as a replacement for polymers derived sources with unreliable supply i.e. from by-products of orange peel oil or turpentine oil.

TABLE 24

| | Control mixture | Fraction 1 test mixture |
|---|---|---|
| Yield | 89% | 83% |
| Isoprene dimers | 4.9% | 3.6% |
| Number average molar | 740 | 775 |
| mass (Mn) | | |
| Mass average molar | 1180 | 1440 |
| mass (Mw) | | |
| Softening point/C. | 112.5 | 114.5 |
| Glass transition | 66 | 68 |
| temperature Tg/C. | | |
| Colour value (APHA) | 150 | 150 |

### Example 32 (Prophetic)

A mixture comprising dimethylcyclooctadiene isomers is produced according to the method of example 31. A fraction comprising various percentage of dimethylcyclooctadienes is prepared by fractional distillation of the mixture. This fractionated composition is then polymerised using the technique of ring opening metathesis polymerisation (ROMP) according to the methods described by Henry Martinez et al. in Polymer Chemistry, issue 11, 2014, Ring Opening Metathesis Polymerisation of 8 membered cyclic olefins.

By this method various ROMP polymers can be produced from sustainably. These would have utility in controlling crystallinity, hydrophobicity and morphology in various types of copolymers.

### Example 33

150 grams of reference standard isoprene were fed into a reactor. The mixture was titrated with ˜0.1 M Butyl Lithium until polymerisation activity was found, then 5 mL of 0.1M BuLi was added. At 50% conversion the reaction was terminated by the addition of methanol. The reaction was “paused” overnight and reinitiated with a titration using the BuLi solution on the next morning, after which 10 mL of BuLi was added. The reaction was allowed to go to completion. A second experiment was performed using the same method but this time using renewable isoprene produced via purification of crude isoprene according to Example 3. Purification processes to obtain polymer grade isoprene are outlined in prior art. The bio-isoprene was purified by distillation and then further treated with molecular sieve 3A, 4A and 5A, then with activated alumina, then with another alumina selexsorb cdx, and finally with silica gel. The purified isoprene had less than 200 ppm of polar compounds and less than 10,000 ppm of C5 hydrocarbons like methyl butenes. As evidenced by the polymerization results below the purified isoprene was suitable for polymerization and behaved identically to fossil isoprene which has stringent specifications for presence of polar impurities down to less than 25 ppm.

Results are shown in Table 25. Comparison between the fossil reference isoprene and the sustainable isoprene produced using the method of Example 3 shows the polymerisation performance can be considered to be broadly equivalent.

TABLE 25

| | Fossil isoprene | Renewable isoprene |
|---|---|---|
| Step 1 | | |
| Isoprene feed (g) | 149.6 | 150.2 |
| BuLi active 1 (mL) | 5 | 5 |
| Isoprene Conversion (%) | 56 | 51.9 |
| MP (g/mole) | 346000 | 314000 |
| Step 2 | | |
| BuLi active 2 (mL) | 10 | 10 |
| Conversion (%) | >99 | >99 |
| MP (g/mole) | 94000 | 89000 |

### Example 34 (Prophetic)

A sample of polymer grade fossil isoprene was mixed with styrene in the ratio 85% isoprene:15% styrene. Using butyl lithium to initiate anionic polymerization a polymer product was produced. This polymer was a styrene-isoprene diblock copolymer.

A sample of bio-isoprene was prepared according to example 3. This material was blended with the polymer grade fossil isoprene in the ratio 1 part fossil-isoprene to 1 part bio-isoprene. Without further purification, this was then further mixed with polymer grade styrene in the ratio 85% isoprene:15% styrene. Using butyl lithium to initiate anionic polymerization, a polymer product was produced.

The distilled bio-isoprene was further treated with molecular sieve 3A, 4A and 5A then with activated alumina (like selexsorb cdx) and finally with silica gel. This purified bioisoprene was used as 100% bio-derived isoprene. The bio-isoprene was mixed with styrene to form a 85% bio-isoprene:15% styrene mixture. This mixture was subjected to anionic polymerization and the resulting product was found to be a mixture of SI diblock copolymer and SIS triblock copolymer.

This example shows that the bioisoprene is suitable for use in the production of block copolymers. It further shows that with purification, the bioisoprene is capable of giving extremely good product yields. It further shows that with appropriate purification the bioisoprene is able to produce useable triblock copolymers.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A rocket fuel composition comprising alicyclic hydrocarbons and optionally aromatic hydrocarbons wherein the total renewable content is at least about 5% w/w, preferably at least about 10% w/w, preferably at least about 15% w/w, preferably at least about 20, preferably at least about 25, preferably at least about 30% w/w of the composition.

2. The composition of claim 1 wherein the alicyclic hydrocarbon content is greater than about 5% w/w, preferably greater than about 10% w/w, preferably greater than about 15% w/w, preferably greater than about 20% w/w, preferably greater than about 25% w/w, preferably greater than about 30% w/w, preferably greater than about 50% w/w, preferably greater than about 70% w/w, preferably greater than about 95% w/w, preferably greater than about 99% w/w.

3. The composition of claim 1 wherein a sulphur content of the composition is less than about 30 ppm, preferably less than about 25 ppm, preferably less than about 20 ppm, preferably less than about 15 ppm, preferably less than about 10 ppm, preferably less than about 5 ppm.

4. The composition of claim 1 wherein a freezing point of the composition is lower than −40 degrees Centigrade.

5. The composition of claim 1 wherein a heat content of the composition is at least about 120000, preferably at least about 121000, preferably at least about 122000, preferably at least about 123000, preferably at least about 124000, preferably at least about 125000 BTU/US gallon at 15 degrees Centigrade.

6. The composition of claim 1 wherein a polyaromatic aromatic hydrocarbon (PAH) content is less than about 3% w/w, preferably less than about 2% w/w, preferably less than about 1% w/w of the composition, preferably less than about 0.01% w/w of the composition.

7. The composition of claim 1 wherein the aromatic hydrocarbons comprises cymene isomers.

8. The composition of claim 1 wherein the alicyclic hydrocarbons comprises alkyl substituted cyclohexanes and/or alkyl substituted cyclooctanes

9. The composition of claim 1 wherein the aliphatic hydrocarbon comprises substantially 10 carbon hydrocarbons at a concentration greater than about 10% w/w, preferably greater than about 20% w/w, preferably greater than about 30% w/w, preferably greater than about 40% w/w, preferably greater than about 50% w/w, preferably greater than about 60% w/w, preferably greater than about 90% w/w.

10. The composition of claim 1 wherein all aromatic hydrocarbon content is less than about 3% w/w, preferably less than about 2% w/w, preferably less than about 1% w/w of the composition, preferably less than about 0.01% w/w of the composition.
